(19) United States
(12) Patent Application Publication
(10) Pub. No.: US 2021/0338806 A1
(43) Pub. Date: Nov. 4, 2021

(54) METHODS OF GENERATING VACCINES AGAINST NOVEL CORONAVIRUS, NAMED SARS-COV-2 COMPRISING VARIABLE EPITOPE LIBRARIES (VELs) AS IMMUNOGENS

(71) Applicant: Primex Clinical Laboratories, Van Nuys, CA (US)

(72) Inventor: Karen Manucharyan, Jardines En La Montana (MX)

(21) Appl. No.: 17/245,355

(22) Filed: Apr. 30, 2021

Related U.S. Application Data
(60) Provisional application No. 63/058,890, filed on Jul. 30, 2020.
(60) Provisional application No. 63/018,814, filed on May 1, 2020.

(51) Int. Cl. A61K 39/215; A61P 31/14; C12N 15/10

(52) U.S. Cl. CPC A61K 39/215; A61K 2039/545; C12N 15/1037; A61P 31/14

(57) ABSTRACT

Described herein is the application of Variable Epitope Libraries (VELs) as immunogens for the generation of vaccines against a novel coronavirus, named SARS-CoV-2. The VELs bearing combinatorial epitope libraries target antigenic variability of viruses such as SARS-CoV-2, and cancer, thus representing a true alternative to traditional vaccine platforms.

### PRIORITY

This application claims the benefit of U.S. provisional applications 63/058,890 filed Jul. 30, 2020, and 63/018,814 filed May 1, 2020, the entire contents of both applications are incorporated by reference herein.

### FIELD

The disclosure relates to compositions and methods for preventing and/or treating diseases associated with the antigenically variable pathogens of SARS-CoV-2.

### BACKGROUND

In 2019, a new disease of unknown etiology appeared in Wuhan, China. Whole virus genome sequences were obtained either directly from patient samples or from cultured viruses from a number of patients hospitalized with pneumonia in Wuhan, showing that the etiological agent was a betacoronavirus belonging to a new clade in subgenus Sarbecovirus in the Orthocoronavirinae subfamily (MacKenzie and Smith 2020, referencing Zhu N., et al. 2020; Zhou P., et al. 2020; Ren L. L. et al. 2020; Lu R., et al. 2020; Wu, F., Zhao, S., Yu, B. et al. 2020). Based on established practice, the new virus was named SARS-CoV-2 by the Coronavirus Study Group of the International Committee for the Taxonomy of Viruses (Gorbalenva A. E. et al. 2020) and the disease it causes was named COVID-19 by WHO (World Health Organization (2020) Novel coronavirus (2019-nCoV). Situation Report 22. 11 Feb. 2020, available on the world wide web at who.int/docs/default-source/coronaviruse/situation-reports/20200211-sitrep-22-ncov.pdf?sfvrsn=fb6d49b1_2 (accessed 22 Feb. 2020).

Coronaviruses are a large family of viruses that usually cause mild to moderate upper-respiratory tract illnesses, like the common cold, in people. However, three times in the 21st century coronavirus outbreaks have emerged from animal reservoirs to cause severe disease and global transmission concerns according to the US National Institutes of Health (NIH) available on the world wide web at niaid.nih.gov/diseases-conditions/coronaviruses. Seven coronaviruses are known to cause human disease, four of which are mild: viruses 229E, OC43, NL63 and HKU1; and three of which can have more serious outcomes in humans: SARS (severe acute respiratory syndrome) which emerged in late 2002 and disappeared by 2004, MERS (Middle East respiratory syndrome), which emerged in 2012 and remains in circulation in camels, and COVID-19, which emerged in December 2019 from China and is caused by the coronavirus known as SARS-CoV-2 available on the world wide web at niaid.nih.gov/diseases-conditions/coronaviruses).

Research directed to developing vaccines against the SARS-CoV-2 virus is ongoing. However, vaccines based on viral-encoded peptides may not be effective against future coronavirus epidemics, as virus mutations could make them ineffective. Indeed, new influenza virus strains emerge every year, requiring immunizations with new vaccines.

Thus, one obstacle in the advancement for developing vaccines against pathogens with genetic variability is immune escape. Typically, immune escape involves amino acid substitutions in peptide epitopes of a pathogenic antigen, the majority of which are not recognized by T cells, particularly the CD8+ class of T cells. This may explain the immune system's failure in clearing or containing various pathogens. The ability of pathogens to escape immunity by mutating amino acids in epitopes or flanking regions (affecting the correct epitope processing) is an ongoing and dynamic process involving complex viral-host interactions. Other factors affecting the immune escape phenomenon include viral fitness, cost of mutations, immune pressure exerted by the host, host genetic factors, and viral load.

One obstacle in treating COVID-19 relates to the genetic variability found in all RNA viruses as the virus mutates over time in a subject and among infected subjects. There is a need in the art for a vaccine to SARS-CoV-2 where the vaccine is effective over time and where mutagenesis of the virus does not decrease effectiveness of the vaccine.

### SUMMARY

The disclosure relates in part to a method of treating and/or preventing disease resulting from viral infection in an subject by the virus SARS-CoV-2, in which a SARS-CoV-2 variable epitope library composition is administered to said subject, the composition comprising one or more synthetic peptide(s), each said peptide comprising either an amino acid sequence identical to an epitope of a SARS-CoV-2 viral antigen or an amino acid sequence which differs from said epitope in at least one corresponding amino acid residue, or nucleic acid encoding said synthetic peptide(s) and a pharmaceutically acceptable excipient. In an embodiment, said one or more peptides are about 7 to about 50 amino acids in length. A peptide variant of a SARS-CoV-2 viral epitope comprises one or more residues which has an amino acid that differs from that of the corresponding one or more residues in the SARS-CoV-2 viral epitope. In an embodiment, from about 1% to about 50% of the total amino acid residues of the peptide variants of a SARS-CoV-2 viral epitope are variable amino acids with respect to their corresponding peptide epitope. Described herein are compositions comprising a peptide SARS-CoV-2 viral epitope(s) and/or corresponding peptide variant(s) of the SARS-CoV-2 viral epitope(s) thereof, preferably comprising a pharmaceutically acceptable excipient, and methods of treatment comprising the compositions.

In one embodiment disclosed herein is a method of generating an immune response in a subject to SARS-CoV-2, the method comprising: administering a SARS-CoV-2 variable epitope library composition comprising a synthetic peptide comprising an amino acid sequence corresponding to an epitope of a SARS-CoV-2 viral epitope, or administering nucleic acid encoding the synthetic peptide, wherein the peptide is 7 to 50 amino acids in length, wherein from 1% to 50% of the total amino acids of the one or more peptides are variable amino acids, and a pharmaceutically acceptable excipient, thereby to generate an immune response to SARS-CoV-2.

In one aspect of the method, the SARS-CoV-2 viral antigen comprises a CTL epitope, the amino acid sequence of the CTL epitope is IVNSVLLFLAFVVFLLVTLAILTAL (SEQ ID NO:1), and the variants of peptide epitope IVNSVLLFLAFVVFLLVTLAILTAL (SEQ ID NO:1), are IVNSVLXFLAFXVFLLVTLXILTAL, (SEQ ID NO:2), wherein the SARS-CoV-2 viral antigen comprises a CTL epitope, and wherein “X” is any of the 20 proteinogenic amino acids the standard genetic code.

In one aspect of the method, the SARS-CoV-2 viral antigen comprises a CTL epitope, the amino acid sequence of the CTL epitope is AILTALRLCAYCCNIVNVSLVKPSFYVY, (SEQ ID NO:3), and the variants of peptide epitope AILTALRLCAYCCNIVNVSLVKPSFYVY, (SEQ ID NO:3), are AILTXLRLCAYXCNIVXVSLVKPXFYVY, (SEQ ID NO:4), wherein “X” is any of the 20 proteinogenic amino acids the standard genetic code.

In one aspect of the method, the SARS-CoV-2 viral antigen comprises a CTL epitope, the amino acid sequence of the CTL epitope is FLWLLWPVTLACFVLAAVYRI, (SEQ ID NO:5), and the variants of peptide epitope FLWLLWPVTLACFVLAAVYRI, (SEQ ID NO:5), are FLWXLXPVTLXCFVLXAVYRI, (SEQ ID NO:6), wherein “X” is any of the 20 proteinogenic amino acids the standard genetic code. In one aspect of the method, the SARS-CoV-2 viral antigen comprises a CTL epitope, the amino acid sequence of the CTL epitope is TVATSRTLSYYKL, (SEQ ID NO:7), and the variants of peptide epitope TVATSRTLSYYKL, (SEQ ID NO:7), are TVXTSRXLSXYKL, (SEQ ID NO:8), wherein “X” is any of the 20 proteinogenic amino acids the standard genetic code.

In one aspect of the method, the SARS-CoV-2 viral antigen comprises a CTL epitope, the amino acid sequence of the CTL epitope is SASAFFGMSRIGMEVTPSGTWLTYTGAIKL, (SEQ ID NO:9), and the variants of peptide epitope SASAFFGMSRIGMEVTPSGTWLTYTGAIKL, (SEQ ID NO:9), are SAXAFXGMSRXGMEVTPSGTWLTYXGXIKL, (SEQ ID NO:10), wherein “X” is any of the 20 proteinogenic amino acids the standard genetic code.

In one aspect of the method, the SARS-CoV-2 viral antigen comprises a CTL epitope, the amino acid sequence of the CTL epitope is YTMADLVYAL, (SEQ ID NO:11), and the variants of peptide epitope YTMADLVYAL, (SEQ ID NO:11), are YTXADXVXAL, (SEQ ID NO:12), wherein “X” is any of the 20 proteinogenic amino acids the standard genetic code.

In one aspect of the method, the SARS-CoV-2 viral antigen comprises a CTL epitope, the amino acid sequence of the CTL epitope is SMMGFKMNY, (SEQ ID NO:13), and the variants of peptide epitope SMMGFKMNY, (SEQ ID NO:13), are SMXGXKXNY, (SEQ ID NO:14), wherein “X” is any of the 20 proteinogenic amino acids the standard genetic code.

In one aspect of the method, the SARS-CoV-2 viral antigen comprises a CTL epitope, the amino acid sequence of the CTL epitope is FLMSFTVLCLTPVY, (SEQ ID NO:15), and the variants of peptide epitope FLMSFTVLCLTPVY, (SEQ ID NO:15), are FLMXFXVLCXTPVY, (SEQ ID NO:16), wherein “X” is any of the 20 proteinogenic amino acids the standard genetic code.

In one aspect of the method, the SARS-CoV-2 viral antigen comprises a CTL epitope, the amino acid sequence of the CTL epitope is KLNDLCFTNVYADSFVIRGDEVRQIAPGQTGKIADYNYKL, (SEQ ID NO:17), and the variants of peptide epitope KLNDLCFTNVYADSFVIRGDEVRQIAPGQTGKIADYNYKL, (SEQ ID NO:17), are KLNDLXFXNVYADSFVIRGDEXRQIAPGQTGKIADXNXKL, (SEQ ID NO:18), wherein “X” is any of the 20 proteinogenic amino acids the standard genetic code.

In one aspect of the method, the SARS-CoV-2 viral antigen comprises a CTL epitope, the amino acid sequence of the CTL epitope is YIWLGFIAGLIAIV, (SEQ ID NO:19), and the variants of peptide epitope YIWLGFIAGLIAIV, (SEQ ID NO:19), are YIWLXFIXGXIAIV, (SEQ ID NO:20), wherein “X” is any of the 20 proteinogenic amino acids the standard genetic code.

In one aspect of the method, the SARS-CoV-2 viral antigen comprises a CTL epitope, the amino acid sequence of the CTL epitope is CVADYSVLYNSASFSTFKCY, (SEQ ID NO:21), and the variants of peptide epitope CVADYSVLYNSASFSTFKCY, (SEQ ID NO:22), are CVADXSXLYNSASFSTXKCY, (SEQ ID NO:22), wherein “X” is any of the 20 proteinogenic amino acids the standard genetic code.

In one aspect of the method, the SARS-CoV-2 viral antigen comprises a CTL epitope, the amino acid sequence of the CTL epitope is FERDISTEIYQAGSTPCNGVEGFNCYFPLQS, (SEQ ID NO:23), and the variants of peptide epitope FERDISTEIYQAGSTPCNGVEGFNCYFPLQS, (SEQ ID NO:23), are FERDISTEXYQXGXTPCNGXEXFNCYFPLQS, (SEQ ID NO:24), wherein “X” is any of the 20 proteinogenic amino acids the standard genetic code.

In one aspect of the method, the SARS-CoV-2 viral antigen comprises a CTL epitope, and wherein the variable amino acids can be any naturally occurring amino acids.

In one aspect of the method, the total number of different peptides or in the library is 87.

In one aspect of the method, the composition is administered to the subject prophylactically.

In one aspect of the method, the composition is administered to the subject prophylactically at a dose from 100 μg to 1 mg of isolated peptides.

In one aspect of the method, one or more doses of the composition are administered to the subject prophylactically at weekly intervals.

In one aspect of the method, the subject has a COVID-19 associated disease and wherein the composition is administered to the subject therapeutically.

In one aspect of the method, the subject has a COVID-19 associated disease and wherein the composition is administered to the subject therapeutically at a dose from 100 μg to 1 mg of isolated peptides.

In one aspect of the method, the subject has a COVID-19 associated disease and wherein one or more doses of the composition are administered to the subject therapeutically at weekly intervals.

In one aspect of the method, the total number of different peptides in the library is from 20 to 8,000.

In one aspect of the method, the variable amino acid variable is any of Alanine, Cysteine, Aspartate, Glutamate, Phenylalanine, Histidine, Isoleucine, Leucine, Asparagine, Glutamine, Arginine, Threonine, Valine or Tryptophan.

In one aspect of the method, the variable amino acid variable is any of Aspartate, Phenylalanine, Isoleucine, Lysine, Leucine, Methionine, Asparagine, Glutamine, Serine, Threonine, Valine or Tyrosine.

In one aspect of the method, the variable amino acid variable is any of Alanine, Aspartate, Glutamate, Phenylalanine, Glycine, Histidine, Isoleucine, Leucine, Asparagine, Proline, Glutamine, Arginine, Serine, Threonine, Valine or Tyrosine.

In one aspect of the method, prophylactically administering the variable epitope library vaccine composition, or nucleic acid encoding the peptides, results in increased proliferation of splenocytes of the subject.

In one aspect of the method, prophylactically administering the variable epitope library vaccine composition or nucleic acid encoding the peptides, results in an immune response comprising an increased number of CD8+IFN-γ+ cells which recognize variant COVID-19-derived CTL epitopes than in the immune response resulting from administering COVID-19 peptides or nucleic acid encoding the peptides.

Also disclosed herein are methods of identifying a set of peptides for the treatment and/or prevention of disease in an individual resulting from infection with or association with SARS-CoV-2. In one embodiment, the set of peptides comprises one or more peptides comprising

(i) a T cell epitope of an antigen expressed in the subject and/or (ii) variants of the T-cell epitope, comprising:

(a) generating a combinatorial variable epitope library (VEL) wherein the VEL comprises a plurality of peptides, each the peptide comprising a T cell epitope or variant thereof, wherein the length of each the T cell epitope or variant thereof, ranges from 8 to 11 amino acids, wherein the amino acid residues at MHC class I-anchor positions of the T cell epitope and its variant are identical, wherein the sequence of the T cell epitope and the variant thereof differ in at least two residues,

(b)

(i) incubating the T cell epitope or a variant thereof, with peripheral blood mononuclear cells (PBMCs) from a healthy subject (or a population of healthy subjects) under conditions suitable for inducing proliferation of PBMCs;

(ii) incubating the T cell epitope or variant thereof, with PBMCs from the subject afflicted with SARS-CoV-2 or condition under conditions suitable for inducing proliferation of PBMCs, wherein the afflicted subject has a MHC Class I haplotype which is similar to the MHC Class I haplotype of the healthy subject,

(iii) comparing the proliferation of the T cell epitope and of each the variant thereof, in step (b)(i) versus step (b)(ii), thereby identifying three peptide groups:
(a) Group I—peptides which induce proliferation of PBMCs of the afflicted subject and in the healthy population
(b) Group II—peptides which induce proliferation of PBMCs of the afflicted subject but not in the healthy population
(c) Group III—peptides which do not induce proliferation of PBMCs of said afflicted subject but induce proliferation in the healthy population

wherein each said peptide Group, or a combination of two or more of Groups I, II, and/or III, identifies a set of peptides for treatment against the disease or condition afflicting said subject. An embodiment of the methods further comprises chemical synthesis of said peptides, optionally wherein the chemical synthesis is performed in the wells of a 96 well plate. In an embodiment of the methods, the sequence of said T cell epitope and its variant(s) thereof differ at only two amino acid residues, the VEL comprises at least 100 variant peptides. In an embodiment of the methods, the sequence of said T cell epitope and its variant(s) thereof differ at only three amino acid residues, the VEL comprises at least 1000 variant peptides. In an embodiment of the methods, the variants are selected randomly. In an embodiment of said methods, the variants are selected semi-randomly. In an embodiment of the methods, the sequence of said T cell epitope is IVNSVLLFLAFVVFLLVTLAILTAL, (SEQ ID NO:1), and in an embodiment of the method, the variants of peptide epitope IVNSVLLFLAFVVFLLVTLAILTAL, (SEQ ID NO:1), are IVNSVLXFLAFXVFLLVTLXILTAL, (SEQ ID NO:2), wherein “X” is any of the 20 proteinogenic amino acids the standard genetic code.

In an embodiment of said methods, the sequence of the CTL epitope is AILTALRLCAYCCNIVNVSLVKPSFYVY, (SEQ ID NO:3), and in an embodiment of said method, the variants of peptide epitope AILTALRLCAYCCNIVNVSLVKPSFYVY, (SEQ ID NO:3), are AILTXLRLCAYXCNIVXVSLVKPXFYVY, (SEQ ID NO:4), wherein “X” is any of the 20 proteinogenic amino acids the standard genetic code. In an embodiment of the methods, the sequence of said CTL epitope is FLWLLWPVTLACFVLAAVYRI, (SEQ ID NO:5), and in an embodiment of the method, the variants of peptide epitope FLWLLWPVTLACFVLAAVYRI, (SEQ ID NO:5), are FLWXLXPVTLXCFVLXAVYRI, (SEQ ID NO:6), wherein “X” is any of the 20 proteinogenic amino acids the standard genetic code. In an embodiment of said methods, the sequence of the CTL epitope is TVATSRTLSYYKL, (SEQ ID NO:7), and in an embodiment of said method, the variants of peptide epitope TVATSRTLSYYKL, (SEQ ID NO:7), are TVXTSRXLSXYKL, (SEQ ID NO:8), wherein “X” is any of the 20 proteinogenic amino acids the standard genetic code. In an embodiment of the methods, the sequence of said CTL epitope is SASAFFGMSRIGMEVTPSGTWLTYTGAIKL, (SEQ ID NO:9), and in an embodiment of the method, the variants of peptide epitope SASAFFGMSRIGMEVTPSGTWLTYTGAIKL, (SEQ ID NO:9), are SAXAFXGMSRXGMEVTPSGTWLTYXGXIKL, (SEQ ID NO:10), wherein “X” is any of the 20 proteinogenic amino acids the standard genetic code. In an embodiment of said methods, the sequence of the CTL epitope is YTMADLVYAL, (SEQ ID NO:11), and in an embodiment of said method, the variants of peptide epitope YTMADLVYAL, (SEQ ID NO:11) are YTXADXVXAL, (SEQ ID NO:12), wherein “X” any of the 20 proteinogenic amino acids the standard genetic code. In an embodiment of the methods, the sequence of said CTL epitope is SMMGFKMNY, (SEQ ID NO:13), and in an embodiment of the method, variants of peptide epitope SMMGFKMNY, (SEQ ID NO:13), are SMXGXKXNY, (SEQ ID NO:14), wherein “X” is any of the 20 proteinogenic amino acids the standard genetic code. In an embodiment of said methods, the sequence of the CTL epitope is FLMSFTVLCLTPVY, (SEQ ID NO:15), and in an embodiment of said method, the variants of peptide epitope FLMSFTVLCLTPVY, (SEQ ID NO:15), are FLMXFXVLCXTPVY, (SEQ ID NO:16), wherein “X” is any of the 20 proteinogenic amino acids the standard genetic code. In an embodiment of the methods, wherein the sequence of said CTL epitope is KLNDLCFTNVYADSFVIRGDEVRQIAPGQTGKIADYNYKL, (SEQ ID NO:17), and in an embodiment of the method, the variants of peptide epitope KLNDLCFTNVYADSFVIRGDEVRQIAPGQTGKIADYNYKL, (SEQ ID NO:17), are KLNDLXFXNVYADSFVIRGDEXRQIAPGQTGKIADX NXKL, (SEQ ID NO:18), wherein “X” is any of the 20 proteinogenic amino acids the standard genetic code.

In an embodiment of said methods, the sequence of the CTL epitope is YIWLGFIAGLIAIV, (SEQ ID NO:19), and in an embodiment of said method, the variants of peptide epitope YIWLGFIAGLIAIV, (SEQ ID NO:19), are YIWLXFIXGXIAIV, (SEQ ID NO:20), wherein “X” is any of the 20 proteinogenic amino acids the standard genetic code. In an embodiment of the methods, the sequence of said CTL epitope is CVADYSVLYNSASFSTFKCY, (SEQ ID NO:21), and in an embodiment of the method, the variants of peptide epitope CVADYSVLYNSASFSTFKCY, (SEQ ID NO:21), are CVADXSXLYNSASFSTXKCY, (SEQ ID NO:22), wherein “X” is any of the 20 proteinogenic amino acids the standard genetic code.

In an embodiment of said methods, the sequence of the CTL epitope is FERDISTEIYQAGSTPCNGVEGFNCYFPLQS, (SEQ ID NO:23), and in an embodiment of said method, the variants of peptide epitope FERDISTEIYQAGSTPCNGVEGFNCYFPLQS, (SEQ ID NO:23), are FERDISTEXYQXGXTPCNGXEXFNCYFPLQS, (SEQ ID NO:24), wherein “X” is any of the 20 proteinogenic amino acids the standard genetic code.

An embodiment of said methods further comprises immunization of the afflicted subject with a formulation comprising at least one or with the mixture of up to 100 variant peptides identified in step (b) and pharmaceutically acceptable carrier.

In an embodiment of the methods, the sets of peptide epitopes of said combinatorial variable epitope library (VEL) are expressed by one or more of the group consisting of plasmid DNA, a viral vector and a microorganism.

In an embodiment of the methods, the sets of peptide epitopes of said combinatorial variable epitope library (VEL) are present at the surface of the microorganism, wherein said microorganism is selected from the group consisting of bacteriophage, yeast and bacteria. In an embodiment of the methods, wherein the sets of peptide epitopes of said combinatorial variable epitope library (VEL), are expressed on the surface of insect cells in combination with an MHC class I molecule. In an embodiment of the methods, wherein said plurality of peptides comprises three or more peptides.

### DESCRIPTION

The methods disclosed herein are useful in COVID-19 therapies associated with SARS-CoV-2 infection and pathogenesis as well as for prophylaxis.

### Definitions

As used herein, a “vaccine” is an immunogen which when applied to a subject, provides the subject with a protective immune responses against disease associated with contact with the pathogen. The protective immune responses generated by an effective vaccine include generating strong and broad cellular and humoral immune responses leading to the generation of cytotoxic lymphocytes (CTLs) and neutralizing antibodies, respectively. Thus, the generation of a protective immune response in a subject who received an effective vaccine against a pathogen can be measured, e.g., by detection in the subject of neutralizing antibodies and cytotoxic lymphocytes that target the pathogen. subsequent contact

An “immune response” in a subject is defined as generation and activation of leukocytes, including but not limited to T cells and B cells, specific for providing protective immunity against SARS-CoV-2. In vitro assays include measuring the T-cell proliferative responses against cells bearing SARS-CoV-2 epitopes as measured by flow cytometry.

“SARS-CoV-2” refers to a coronavirus 2 virus whose infection causes severe acute respiratory syndrome. SARS-CoV-2 is a betacoronavirus which is believed to have its origin, at least in part, in bats. In humans, the SARS-CoV-2 virus causes coronavirus disease 2019 (COVID-19). Common symptoms of a COVID-19 infection in humans include fever, tiredness and dry cough.

A “variable epitope library” (VEL) comprises peptide immunogens comprising a peptide epitope and/or one or more peptide variants of the peptide epitope. Preferably, a VEL comprises a peptide epitope and numerous peptide variants of the epitope, for example, up to 10², 10³, 10⁴, 10⁵, 10⁶, 10⁷, 10⁸, 10⁹ or 10¹⁰ peptide variants of the epitope, or more. A peptide variant of a SARS-CoV-2 viral epitope comprises one or more residues which has an amino acid that differs from that of the corresponding one or more residues in the SARS-CoV-2 viral epitope. Peptide variants are immunogenic peptides which have the potential to protect a subject against a pathogen with a high mutation rate, such as an RNA virus, for example, where one or more of the residues of an epitope of the pathogen mutates over time, thus giving rise to a variant of the epitope which has a modified amino acid sequence relative to the amino acid sequence of the epitope. If a subject has been treated with a VEL library that comprises a peptide variant which has the sequence of the mutated epitope and has developed an immune response directed to the mutated epitope, then upon primary exposure to the pathogen with the altered peptide epitope, the subject may have already developed some degree of immunity to the pathogen with the altered peptide epitope as a result of previous treatment with a VEL library comprising the specific peptide altered in the pathogen.

From about 1% to about 50% of the total amino acid residues of the peptide variants of a SARS-CoV-2 viral epitope are variable amino acids with respect to their corresponding peptide epitope.

A VEL may contain up to and including 10¹, 10², 10³, 10⁴, 10⁵, 10⁶, 10⁷, 10⁸, 10⁹, 10¹⁰, 10¹¹, 10¹², 10¹³ or more peptides or nucleic acid molecules encoding said peptides. VEL library is a collection of synthetic peptides or a collection of nucleic acids encoding the synthetic peptides. The synthetic peptides comprise a peptide epitope of an antigen, and peptide variants of the peptide epitope. The amino acid sequence of the peptide variants corresponds to the amino acid sequence of the peptide epitope

An “epitope” is a portion of an antigen recognized by a cell of the immune system, including but not limited to a B cell and a T cell.

A “cytotoxic T lymphocyte (CTL) epitope” is an epitope that is recognized by a cytotoxic T cell. A CTL epitope may be about 7-10 amino acids in length.

A peptide variant of a CTL epitope may be 7-10 amino acids in length, 8-10 amino acids, or 9 amino acids in length.

A SARS-CoV-2 epitope may be an epitope that is recognized by a T helper cell. A “T helper cell epitope” is generally 10-50 amino acids in length.

A peptide that mimics a T helper cell epitope may be 10-50 amino acids in length, 12-30 amino acids, 9-22 amino acids in length, or 13-17 amino acids in length.

A “peptide”, as used herein, has a number of amino acid positions, for example, one such peptide may be composed of 10 amino acids and will therefore have 10 amino acid positions. Specific positions of such a peptide are invariant and other positions are variant and designated “X”. An “invariant position” contains an amino acid which is identical to the amino acid at the corresponding position of an epitope. A “variant position” contains an amino acid whose identity is different from the amino acid at the corresponding position of the same epitope.

A “SARS-CoV-2 variable epitope library” (SARS-CoV-2 VEL) contains a plurality of peptides and/or nucleic acids that encode said peptides, where the peptides are peptide epitope(s) of SARS-CoV-2 and/or variants of the SARS-CoV-2 peptide variants.

A “variable amino acid” SARS-CoV-2 refers is any amino acid, preferably, but not limited to natural amino acids as described herein, which resides at a specified residue location of the peptide epitope. A variant of an epitope is an epitope comprising a variable amino acid at one or more residue positions of the peptide epitope. As described above, up to 10%, up to 20%, up to 30% or up to 40% or up to 50% of amino acid positions within the peptide epitope are replaced by one of the 20 natural amino acids at each amino acids.

Thus, a variable epitope library (VEL) can act as a vaccine to generate an immune response against the SARS-CoV-2 pathogen, as well SARS-CoV-2 genetic/antigenic variants that arise via mutation of the virus during infection and during passage among subjects.

The term “about”, when used herein in reference to a value, refers to a value that is similar, in context to the referenced value. In general, those skilled in the art, familiar with the context, will appreciate the relevant degree of variance encompassed by “about” in that context. For example, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, administration may be ocular, oral, parenteral, topical, etc. Administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (e.g., by intratracheal instillation), vaginal, vitreal, etc. Administration may involve only a single dose. Administration may involve application of a fixed number of doses. Administration may involve dosing that is intermittent (e.g., a plurality of doses separated in time) and/or periodic (e.g., individual doses separated by a common period of time) dosing. Administration may involve continuous dosing (e.g., perfusion) for at least a selected period of time.

As used herein “animal” refers to any member of the animal kingdom. The term “animal” refers to humans, of either sex and at any stage of development. The term “animal” refers to non-human animals, at any stage of development. The non-human animal is a mammal (e.g., a rodent, a mouse, a rat, a rabbit, a monkey, a dog, a cat, a sheep, cattle, a primate, and/or a pig). Animals include, but are not limited to, mammals, birds, a human subject or subject. An animal may be a transgenic animal, genetically engineered animal, and/or a clone.

It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

As used herein, the term “corresponding to” may be used to designate the position/identity of a structural element in a compound or composition through comparison with an appropriate reference compound or composition. For example, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190.sup.th amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GL SEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

As used herein, “epitope” refers to a portion of an antigen that is specifically recognized by an immunoglobulin (e.g., antibody or receptor) binding component. An epitope is comprised of a plurality of chemical atoms or groups on an antigen. Such chemical atoms or groups can surface-exposed when the antigen adopts a relevant three-dimensional conformation. Such chemical atoms or groups are physically near to each other in space when the antigen adopts such a conformation. At least some such chemical atoms are groups are physically separated from one another when the antigen adopts an alternative conformation (e.g., is linearized).

As used herein, the term “isolated” refers to a substance and/or entity that has been (1) separated from at least some of the components with which it was associated when initially produced (whether in nature and/or in an experimental setting), and/or (2) designed, produced, prepared, and/or manufactured by the hand of man Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%), about 97%), about 98%, about 99%, or more than about 99% of the other components with which they were initially associated. Isolated agents are about 80%, about 85%, about 90%, about 91%, about 92%, about 93%, about 94%, about 95%, about 96%, about 97%), about 98%, about 99%, or more than about 99% pure. As used herein, a substance is “pure” if it is substantially free of other components. As will be understood by those skilled in the art, a substance may still be considered “isolated’ or even “pure”, after having been combined with certain other components such as, for example, one or more carriers or excipients (e.g., buffer, solvent, water, etc.); percent isolation or purity of the substance is calculated without including such carriers or excipients. To give but one example, a biological polymer such as a polypeptide or polynucleotide that occurs in nature is considered to be “isolated’ when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, a polypeptide that is chemically synthesized or is synthesized in a cellular system different from that which produces it in nature is considered to be an “isolated’ polypeptide. Alternatively or additionally, a polypeptide that has been subjected to one or more purification techniques may be considered to be an “isolated’ polypeptide to the extent that it has been separated from other components a) with which it is associated in nature; and/or b) with which it was associated when initially produced.

As used herein, the term “pharmaceutical composition” refers to a composition in which an active agent is formulated together with one or more pharmaceutically acceptable carriers. The composition is suitable for administration to a human or animal subject. The active agent is present in unit dose amount appropriate for administration in a therapeutic regimen that shows a statistically significant probability of achieving a predetermined therapeutic effect when administered to a relevant population.

The term “polypeptide”, as used herein, generally has its art-recognized meaning of a polymer of at least three amino acids. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. Proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. Proteins are antibodies, antibody fragments, biologically active portions thereof, and/or characteristic portions thereof.

“Prevent” or prevention: as used herein when used in connection with the occurrence of a disease, disorder, and/or condition, refers to reducing the risk of developing the disease, disorder and/or condition and/or to delaying onset and/or severity of one or more characteristics or symptoms of the disease, disorder or condition. Prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition.

As used herein, the term “specific binding” refers to an ability to discriminate between possible binding partners in the environment in which binding is to occur. A binding agent that interacts with one particular target when other potential targets are present is said to “bind specifically” to the target with which it interacts. Specific binding is assessed by detecting or determining degree of association between the binding agent and its partner; specific binding is assessed by detecting or determining degree of dissociation of a binding agent-partner complex; specific binding is assessed by detecting or determining ability of the binding agent to compete an alternative interaction between its partner and another entity. Specific binding is assessed by performing such detections or determinations across a range of concentrations.

As used herein, the term “subject” refers an organism, typically a mammal (e.g., a human, including prenatal human forms). A subject can be suffering from a relevant disease, disorder or condition. A subject can be susceptible to a disease, disorder, or condition. A subject can display one or more symptoms or characteristics of a disease, disorder or condition. Or a subject does not display any symptom or characteristic of a disease, disorder, or condition. Or a subject is someone with one or more features characteristic of susceptibility to or risk of a disease, disorder, or condition. A subject can be a patient. Or a subject is an individual to whom diagnosis and/or therapy is and/or has been administered.

As used herein, the phrase “therapeutic agent” in general refers to any agent that elicits a desired pharmacological effect when administered to an organism. An agent is considered to be a therapeutic agent if it demonstrates a statistically significant effect across an appropriate population. The appropriate population may be a population of model organisms. An appropriate population may be defined by various criteria, such as a certain age group, gender, genetic background, preexisting clinical conditions, etc. A therapeutic agent can be a substance that can be used to alleviate, ameliorate, relieve, inhibit, prevent, delay onset of, reduce severity of, and/or reduce incidence of one or more symptoms or features of a disease, disorder, and/or condition. A “therapeutic agent” can be an agent that has been or is required to be approved by a government agency before it can be marketed for administration to humans. A “therapeutic agent” can be an agent for which a medical prescription is required for administration to humans.

As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. A therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular subject. Rather, a therapeutically effective amount may be that amount that provides a particular desired pharmacological response in a significant number of subjects when administered to patients in need of such treatment. For example, the term “therapeutically effective amount”, refers to an amount which, when administered to a subject in need thereof in the context of inventive therapy, will block, stabilize, attenuate, or reverse a disease or disorder occurring in said subject. Those of ordinary skill in the art will appreciate that, a therapeutically effective amount may be formulated and/or administered in a single dose. A therapeutically effective amount may be formulated and/or administered in a plurality of doses, for example, as part of a dosing regimen.

As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. A variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. A variant polypeptide or nucleic acid may differ from a reference polypeptide or nucleic acid as a result of one or more differences in amino acid or nucleotide sequence. A variant polypeptide or nucleic acid may show an overall sequence identity with a reference polypeptide or nucleic acid that is at least 85%, 86%, 87%, 88%, 89%, 90%, 91%, 92%, 93%, 94%, 95%, 96%, 97%, or 99%. A variant polypeptide or nucleic acid does not share at least one characteristic sequence element with a reference polypeptide or nucleic acid. A reference polypeptide or nucleic acid has one or more biological activities. A variant polypeptide or nucleic acid shares one or more of the biological activities of the reference polypeptide or nucleic acid.

As used herein, the term “vector” refers to a nucleic acid molecule capable of transporting another nucleic acid to which it has been linked. One type of vector is a “plasmid’, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2.sup.nd ed., Cold Spring Harbor Laboratory Press, Cold Spring Harbor, N.Y. (1989)), which is incorporated herein by reference for any purpose.

The use of the word “a” or an when used in conjunction with the term “comprising” in the claims and/or the specification may mean “one,” but it is also consistent with the meaning of “one or more,” “at least one,” and “one or more than one.” The use of the term or in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the feature in the context with which it is referred. The term “substantially” when referring to an amount, extent or feature (e.g., “substantially identical” or “substantially the same”). It includes a disclosure of “identical” or “the same” respectively, and this provides basis for insertion of these precise terms into claims. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term or combinations thereof as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

### BRIEF DESCRIPTION OF THE DRAWINGS

FIG. 1 Portrays a map of China displaying location and prevalence of COVID-19 confirmed cases as of Feb. 4, 202. Also embedded in FIG. 1 adjacent to the map, is a table displaying the most frequent haplotypes identified for the Chinese population, A*11 and A*02. We chose these two for the vaccine design due to the published scientific literature from China where they showed to be the most frequent in over 200,000 Chinese inhabitants from different provinces as well as of different ethnicities (Pan Q. et al.; Li X. F. et al.; Zhou X. Y. et al.; Shao L. N. et al.).

FIG. 2 Vaccine Selection Based on SARS-CoV-2 proteins.

Genome sequence data from patients infected with SARS-CoV-2 and available protein sequence data were used to identify vaccine candidates. Multi-epitopic regions from SARS-CoV-2 proteins were identified using Immune Epitope Database (IEDB) computational software (https://www.iedb.org/). Publicly available Major Histocompatibility Complex (MHC) data was used to identify epitopes for HLA-A*02:01 and HLA-A*02:11 haplotypes. In this manner 12 vaccine immunogens for SARS-CoV-2 were selected and generated utilizing our variable epitope libraries (VEL) vaccine platform.

FIG. 3 Application of variable epitope libraries (VEL)-based vaccine immunogens as an alternative approach for the development of vaccines against antigenic variable pathogens (AVPs) and cancer. An intact immune system responds upon infection with AVPs and against cancer by generating a limited pool of T cells. Vaccination with defined antigen sequence immunogens (DASIs) induces larger repertoire of lymphocytes, however, these cells were shown to fail to provide protection against APVs and cancer. Vaccines based on VELs generate the largest pool of T cells capable of containing AVPs infection and the development of cancer Van Regenmortel M H. 2014; Bhiman J. N. et al. (2015). TE, TCM, TEM, TRM stand for effector T cells, central, effector and resident memory T lymphocytes, respectively. DASI stands for defined antigen sequence immunogen. AVP stands for antigenically variable pathogen. VEL stands for variable epitope library.

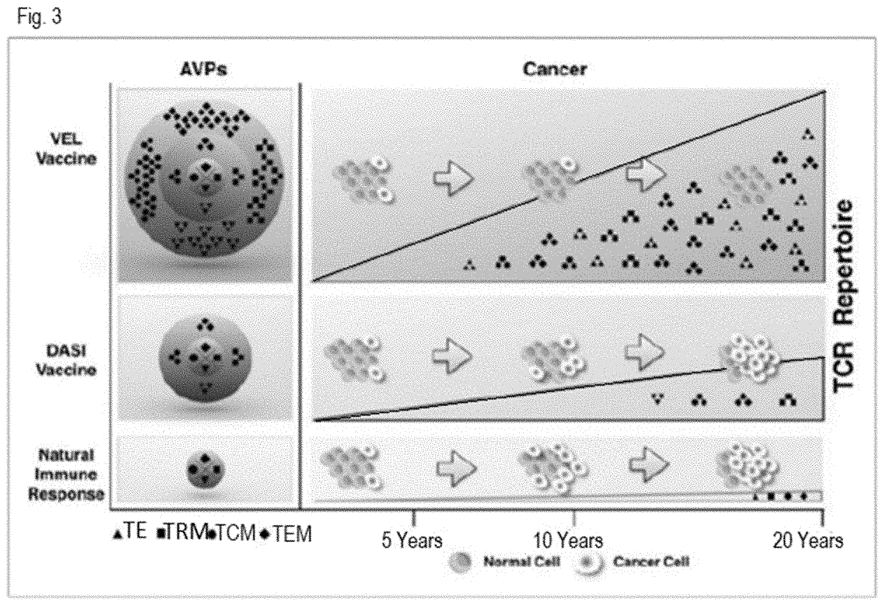

### DETAILED DESCRIPTION

The present disclosure relates to compositions and methods for targeting the antigenically variable pathogen of SARS-CoV-2. Certain embodiments disclosed herein relate to construction of variable epitope libraries (VELs) containing mutated versions of epitopes derived from antigens associated with SARS-CoV-2 for treating subjects in both therapeutic and prophylactic settings. Given the dynamic and elusive nature of antigenic variability of the SARS-CoV-2 virus, there is a need to develop compositions and methods for targeting various SARS-CoV-2 antigenic epitopes to counteract immune escape and provide alternative treatments to these conditions.

Embodiments of the present disclosure provide for VEL compositions and methods of use for treatment of disease. In certain embodiments, a composition may include a synthetic peptide. In accordance with these embodiments, the synthetic peptide may include at least one epitope of a SARS-CoV-2 pathogen-specific polypeptide, where at least one amino acid residue of the peptide is substituted with each of the other nineteen common amino acid residues. In another embodiment, the present disclosure provides for VEL compositions that can include nucleic acid sequences or nucleic acid sequence variations. In accordance with this embodiment, the nucleic acid sequences or nucleic acid sequence variations may encode a peptide having at least one epitope of a pathogen- or disease-specific polypeptide, where at least one amino acid residue of the encoded peptide is substituted with each of the other nineteen common amino acid residues.

In one example, VEL compositions disclosed herein may be prepared by expression in a bacterial, viral, phage display, or eukaryotic expression system. In another example, the VEL compositions may be expressed and displayed on the surface of a recombinant bacteriophage, bacterium or yeast cell. In accordance with these embodiments, the composition of an epitope of a pathogen-specific nucleic acid or polypeptide disclosed herein may be selected from one or more epitopes of SARS-CoV-2.

In another embodiment, a method for preparing and using a variable epitope library may include preparing the variable epitope library (VEL), injecting the library into a subject and inducing an immune response in the subject against the VEL. In accordance with this embodiment, preparing a VEL may include preparing a VEL bearing epitopes of a SARS-CoV-2-specific polypeptide. In one example, inducing an immune response in a subject may include inducing an immune response effective to protect a subject against infection with a SARS-CoV-2 pathogen. In another example, inducing the immune response may include inducing the immune response effective to treat a subject infected with SARS-CoV-2 or to protect the subject against infection by SARS-CoV-2.

Disclosed herein are methods of the treatment and/or prevention of a disease or disorder in a subject resulting from or association with infection with the coronavirus SARS-CoV-2. In one embodiment the disease is COVID-19. In an embodiment, the disease or disorder associated with or resulting from infection with the coronavirus SARS-CoV-2 includes but is not limited to cough, fever, tiredness and difficulty breathing.

VEL libraries and compositions thereof disclosed herein can be administered to a subject prophylactically or therapeutically to treat, prevent, and/or reduce the risk of developing various diseases, e.g., COVID-19, from various pathogens, such as a SARS-CoV-2. Methods disclosed herein can include methods of treating COVID19 in a subject including injecting a variable epitope library vaccine composition having one or more isolated peptides with amino acid sequences corresponding to a one of above CTL epitope, the one or more peptides having from about 7 to about 50 total amino acids, wherein from about 1% to about 50% of the total amino acids of the one or more peptides are variable amino acids, and a pharmaceutically acceptable excipient and/or adjuvant. In accordance with these embodiments, when introduced to a subject, these compositions can generate an immune response. Methods disclosed herein include treating a subject diagnosed with COVID-19 with one or more above VEL compositions, whereby administration of the composition to the subject prevents and/or treats symptoms of COVID-19.

Antigenic Variability

Current licensed vaccines, almost exclusively antibody-based in their action, are protective against pathogens with low antigenic variability (examples include vaccines against diphtheria, tetanus, hepatitis A, hepatitis B, measles, mumps, or rubella viruses). Ref 1. Page 2640, column 1 [1,5,6] However, the common feature of many important pathogens (e.g., COVID-19, human immunodeficiency virus (HIV), hepatitis C virus (HCV), dengue virus (DENV), influenza virus, Ebola virus, Plasmodium species, etc.) is their antigenic variability caused by high mutation rate and/or genetic instability which, in turn, represents an obstacle for the development of effective vaccines.

Mutations happen randomly and are part of the lifecycle. Some mutations will break the virus. Other mutations can benefit it. The mutation rate of COVID-19 appears to be about 24 mutations per year (Bedford, T. (2020), which is similar to other RNA viruses like flu and is equivalent to a mistake every second or third transmission. This coronavirus has a longer genome than flu, so there appear to be fewer mutations per base.

A phenomenon not analyzed systematically in current efforts to generate vaccines against pathogens having antigenic variability is the reduction of antibody and T-cell responses to novel antigenic determinants which develop in the second strain from mutations in the first strain and, consequently, impairs the development of immune memory upon sequential exposure to closely related pathogen variants, Klenerman P. et al (1998) and Kim et al. (2012). The methods described herein avoid that problem by using a Variable Epitope Library to simultaneously expose a subject to both (i) epitope(s) currently expressed by the pathogen as well as (ii) potential mutations of that epitope(s) that may develop in the pathogen in the future. Thus, the immune system has the potential to build immunity to future variations of pathogens having antigenic variability. The simultaneous exposure of both the epitope and it mutations through a Variable Epitope Library as described herein avoids the immunosuppression of immune responses upon sequential later exposure of a mutant of a pathogen having antigenic variability.

There is compelling evidence that CD8+ T cells are key components of immune response against many intracellular pathogens e.g., viruses, and, therefore, effective vaccines against antigen variable pathogens will likely need to induce broad and potent cellular immune responses. The observation that whole protein antigens (Ags) are not necessarily essential for inducing protective immunity has led to the emergence of “structural vaccinology.” Structure-based vaccines are designed on the rationale that suitable epitopes (preferably multiple epitopes) are sufficient to induce protective immune responses against pathogens, including antigenically variable pathogens (AVPs).

An epitope, also referred to as an antigenic determinant, is a portion of an antigen that is recognized by various molecules and cells that make up a subject's immune system (e.g., antibodies, T cells, B cells).

A T-cell epitope of is a specific region of the antigen to which a T cell binds. T-cell epitopes of for example, a protein of an intracellular pathogen, are generated as a result of intracellular processing of the pathogen's proteins by the host and presented as short peptides on the surface of the host's antigen-presenting cells by situating themselves in a pocket of the extracellular domain of a transmembrane protein of the host major histocompatibility complex (MHC). An immune response in the host is initiated following recognition of the epitopes of the pathogen presented in the context of an MHC protein on antigen presenting cells by T cells through the extracellular domains of the T cell receptor (TCR) in the context of the T cell receptor complex.

Thus, epitope recognition in MHC-restricted T-cell responses involves 2 different binding events: first, small peptides bind to the MHC molecules after Ag processing; then, the resulting peptide-MHC (pMHC) complex is bound by T-cell receptor (TCR) leading to cell activation. Current estimates of human ab TCR diversity suggest that there are <10⁸ different Ag receptors in the naive T cell pool which should recognize >10¹⁵ potential peptide MHC complexes.

Personalized vaccines disclosed herein evaluate the interaction or recognition between receptors on the surface of a subject's T cells and a cell surface complex comprising an epitope and a Major histocompatibility protein (MHC). In developing personalized vaccines, a number of factors are considered, including the MHC alleles of a subject, the peptide epitopes generated by the subject, and the T cell repertoire displayed by the subject.

MHC Class I and Class II Polymorphisms

As is well known in the art, there are two different classes of MHC molecules known as MHC class I and MHC class II, which deliver peptides from different cellular compartments to the surface of the infected cell. Peptides from the cytosol are bound to MHC class I molecules which are expressed on the majority of nucleated cells and are recognized by CD8+ T cells. MHC class II molecules, in contrast, traffic to lysosomes for sampling endocytosed protein antigens which are presented to the CD4+ T cells (Bryant and Ploegh, 2004).

Also well known in the art is that peptide epitopes ranging from about 8-11 amino acids bind MHC class I molecules, while large peptide epitopes bind MHC Class II molecules Claus Lundegaard et al. Human MHCs molecules, otherwise known as Human leukocyte antigens (HLA), are highly polymorphic (>2300 human MHC class I molecules encoding HLA-A and -B alleles have been registered by hla.alleles.org (http://hla.alleles.org/nomenclature/stats.html) and most of the polymorphisms influence the peptide binding specificity. As a result of this specificity for peptides displayed by individual alleles of MHC molecules, a specified peptide epitope may bind a MHC Class I molecule of a first subject but not bind a MHC Class I molecule of a second subject. However, MHC alleles can be clustered into supertypes because many allelic molecules have overlapping peptide specificities which are not always obvious from the sequence similarity, as some alleles with very similar HLA sequences will have different binding motifs and vice versa.

Generation of Peptide Epitopes

As is taught in the art, proteins expressed within a cell, including proteins (antigens) from intracellular pathogens or tumor associated antigens, are degraded in the cytosol by a protease complex, the proteasome, which digests polypeptides into smaller peptides, Claus Lundegaard et al., ibid. The protease is a multi-subunit particle, the beta ring of which contains three active sites, each of which is formed by a different subunit: B1, B2 and B5, each of which has different specificities, cleaving preferentially on the carboxylic side of either hydrophobic residues (B5), basic residues (B1), or acidic ones (B2), respectively. In certain cells, or in the presence of gamma interferon, these subunits may be replaced by an alternate set of active site subunits (B1i/LMP2, B2i/MECL1, B5i/LMP7) which results in the production of a different set of peptides, For a review see Rock et al 2010. Thus, the set of proteasome cleaved peptides generated by a cell varies depending on the cell type and/or its environment.

As is taught in the art, a subset of the proteasome-cleaved peptides is bound by the transporter associated with antigen presentation (TAP), Claus Lundegaard et al., ibid, for example. These TAP associated peptides are translocated into the endoplasmic reticulum where, depending on their length and amino acid sequence, they bind MHC class I molecules and are exported as a peptide: MHC class I complex to the cell surface. Thus, the surface of a subject's cells displays a unique distribution of peptide: MHC class I complexes. The cell surface peptide: MHC class I complex is available for recognition by a T cell receptor from the subject's repertoire of T cell receptors displayed on the surface of Cytotoxic T lymphocytes (CTLs).

CTL Recognition of Peptides Associated with MHC Class I

As is taught in the art, Cytotoxic T lymphocytes (CTL)s detect infected or transformed cells by means T cell receptors on the surface of CD8+ T cells which recognize peptide epitopes bound and presented by one of three pairs of cell surface MHC class I molecules (e.g., human HLA-A, HLA-B, and HLA-C molecules). Recognition of a specified peptide epitope depends on many factors, including the ability of the peptide epitope to bind a subject's MHC class I molecule as discussed above, and the presence in a subject's T cell repertoire of CD8+ T cells having a cell surface T cell receptor able to recognize and interact with the cell surface [peptide epitope: MHC class I] complex. It is estimated that for an effective immune response, at least one T cell in a few thousand must respond to a foreign epitope, Mason D. (1998).

T Cell Repertoires Differ Among Subjects

The TCR repertoire of a subject is distinct from that of other subjects as a result of both genetic differences and TCR dependent differences in processing of TCR bearing T-cells. As is taught in the art, the antigen recognition portion of the T cell receptor (TCR) has two polypeptide chains, α and β, of roughly equal length. Both chains consist of a variable (V) and a constant (C) region. The V regions of each pair of chains of a TCR interact with the MHC-peptide complex. Each TCR V region is encoded by one of several V region gene segments (more than 70 human TCR Vα genes and more than 50 human Vβgene segments) which has rearranged with a Jα gene segment to encode the TCR α chain, and both a D and a Jβ gene segment to encode the TCR β chain. McMahan R H, et al. 2006; Wooldridge L, et al., 2011; Parkhurst M R, et al. 1996; Borbulevych O. Y., et al. 2005; Zaremba S., et al., 1997; Salazar E, et al., 2000. The TCR Vα and TCR β gene segments display considerable polymorphism, with many being situated in coding/regulatory regions of functional TCR genes and several causing null and nonfunctional mutations. Gras et al. (2010).

Thus, at least one component of the uniqueness of a subject's T cell repertoire is thought to originate at a genetic level, due to at least in part to any of the polymorphism of T cell receptor loci, with the additional components of imprecise rearrangement of V region gene segments and N and P region addition.

As is taught in the art, clonal selection of lymphocytes expressing T cell receptors with particular antigenic specificities further individualizes a person's T cell repertoire. Birnbaum M E., et al., (2014); Hoppes et al., (2014); Abdul-Alim C. S. et al., (2010); Ekeruche-Makinde et al. (2010) Buhrman et al., (2013); Kappler J. W. et al. (1987); Hengartner H. et al., (1988); Pircher H. et al., (1991).

Though not bound by theory, clonal selection is thought to further selectively refines an already unique set of T cells based on affinity to self-proteins, the self-proteins containing multiple polymorphisms between subjects. The combination of T cell receptor variability at the genomic level, and subsequent clonal selection of the T cells based on the expressed T-cell receptor, and environmental influences thereon, are thought to contribute in providing a T-cell repertoire with a range of binding specificities that is unique to each subject.

It is estimated in the art that a single T cell receptor can recognize more than a million peptides, giving rise to significant T-cell cross reactivity, Wooldridge L, et al. 2011. Epitope variants contain amino acid substitutions in the peptide sequence of an epitope that can improve peptide binding affinity for the MHC (Parkhurst M. R., et al. 1996; Borbulevych O Y, et al. 2005;) and/or alter the interaction of the [peptide-MHC Class I] complex, (Jonathan D. Buhrman and Jill E. Slansky, 2013; McMahan R H, et al. 2006; Zaremba S, et al. 1997; Salazar E, et al. 2000).

Thus, identifying which set of peptides comprising epitopes and variants thereof, are able to bind the specific cell surface MHC class I molecules of a given subject and subsequently interact with the unique repertoire of CTLs present in the given subject at a given time is critical in developing personalized vaccines and/or subjectized immunotherapy directed against intracellular antigens such as those generated by SARS-CoV-2.

Methods are disclosed herein which identify peptides comprising CD8+ T-cell epitopes and/or mimotopes and/or variants thereof, from combinatorial epitope and/or mimotope libraries, using screening assays based on in vitro lymphoproliferation of CD8+ T-cells. From these libraries, sets of randomly selected individual peptides are obtained, preferably using chemical synthesis. These peptides are then applied to various assays to test the ability of the peptides to induce proliferation of peripheral blood mononuclear cells of individual hosts. Conventional assays utilized to detect T cell responses include proliferation assays well known in the art including, but not limited to, lymphokine secretion assays, direct cytotoxicity assays, and limiting dilution assays, for example.

MHC Alleles in the Chinese Population

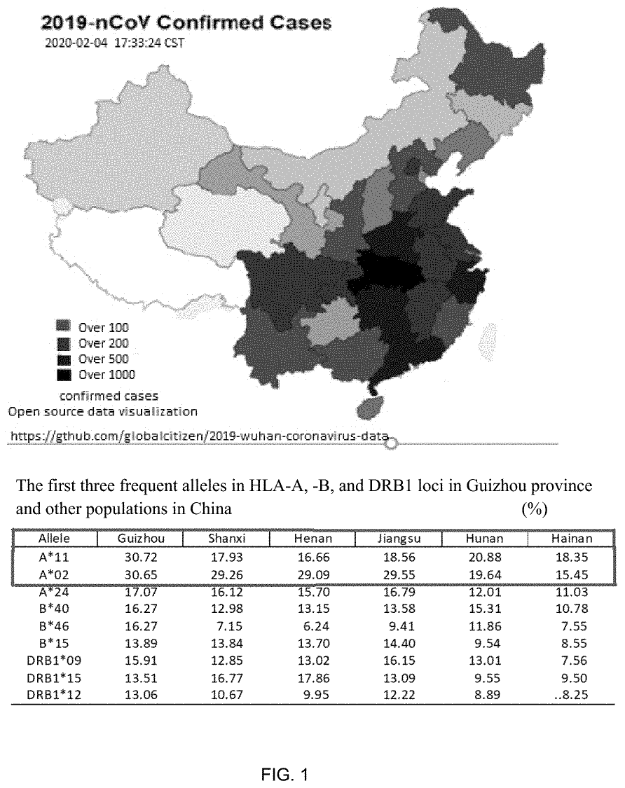

The Table embedded in FIG. 1 displays the most frequent haplotypes identified for the Chinese population, A*11 and A*02. Applicant chose these two haplotypes for the vaccine design. The selection of these two haplotypes was based on published scientific literature from China showing the two haplotypes to be the most frequent in over 200,000 Chinese inhabitants from different provinces as well as of different ethnicities (Pan, Q. et al.; Li, X. F. et al., Zhou, X. Y. et al.).

In one embodiment, methods are disclosed herein which identify a set of peptides for treatment against a disease or condition afflicting a subject, wherein the subset of peptides comprises (i) a T cell epitope of an antigen expressed in said subject and/or (ii) variants of said T-cell epitope, comprising:

(a) generating a combinatorial variable epitope library (VEL) wherein said VEL comprises a plurality of peptides, each said peptide comprising a T cell epitope or variant thereof, wherein the length of each said T cell epitope or variant thereof, ranges from 8 to 11 amino acids, wherein the amino acid residues at MHC class I-anchor positions of said T cell epitope and its variant are identical, wherein the sequence of said T cell epitope and said variant thereof differ in at least two residues,

(i) incubating said T cell epitope or a variant thereof, with peripheral blood mononuclear cells (PBMCs) from a healthy subject (or a population of healthy subjects) under conditions suitable for inducing proliferation of PBMCs;

(ii) incubating said T cell epitope or variant thereof, with PBMCs from said subject afflicted with said disease or condition under conditions suitable for inducing proliferation of PBMCs, wherein said afflicted subject has a MHC Class I haplotype which is similar to the MHC Class I haplotype of said healthy subject,

(iii) comparing the proliferation of said T cell epitope and of each said variant thereof, in step (b)(i) versus step (b)(ii), thereby identifying three peptide groups:

(a) Group I—peptides which induce proliferation of PBMCs of said afflicted subject and in said healthy population;

(b) Group II—peptides which induce proliferation of PBMCs of said afflicted subject but not in said healthy population; and

(c) Group III—peptides which do not induce proliferation of PBMCs of said afflicted subject but induce proliferation in said healthy population

wherein each said peptide Group, or a combination of two or more of Groups I, II and III, identifies a set of peptides for treatment against said disease or condition afflicting said subject.

The epitope is preferably mutated to produce libraries, including combinatorial libraries, preferably by random, semi-random or, in particular, by site-directed random mutagenesis methods, preferably to exchange residues other than the Anchor positions of the MHC Class I T cell epitope. Anchor positions are very restricted in the choice of amino acids and are typically located at residues #2 and 3, near N-terminal end, and positions #8, 9, 10 or 11, near COOH-terminal end of a MHC Class I T cell peptide epitope or mimotope, or variant thereof.

SARS-COV-2 Variable Epitope Library Compositions

Genetic variability of many pathogens and disease-related antigens such as SARS-CoV-2, can result in the selection of mutated epitope variants able to escape control by immune responses. This can be a major obstacle to vaccine development against certain pathogens high genetic variability typical of RNA viruses, e.g., such as SARS-CoV-2. Embodiments herein relate to immunogens composed of variable epitope libraries (VELs) derived from the viral pathogen SARS-CoV-2, in order to advance strategies for overcoming disease and disorders associated with this antigenically variable pathogenic SARS-CoV-2 virus.

A SARS-CoV-2 variable epitope library (VEL) composition comprising at least one SARS-CoV-2 T-cell epitope and its variants. A VEL composition comprises peptides that can be about 7 to about 50 or amino acid residues in length, a length suitable for presentation of the peptides on the cell surface by MHC Class I and II proteins to a T cell receptor or other receptor of an immune cell such as an NK cell. For example, epitope recognition in MHC-restricted T-cell responses involves two different binding events: first, small peptides (epitopes) bind to the extracellular domain of a MHC transmembrane protein after intracellular processing of the antigen protein into small peptides; then the resulting peptide-MHC (pMHC) complex is bound by T-cell receptor (TCR) leading to activation of the T-cell and subsequent generation of an immune response specific to the epitope.

In the case of a variable epitope library composition, the peptides are synthetic and include variants of a peptide epitope. Alternatively, a variable epitope library can contain one or more Class I (CTL) epitopes and their respective variants, and/or one or more Class II (TH) epitopes and their respective variants. Alternatively, a of a variable epitope library can comprise a library of nucleic acids encoding said peptides as described herein.

For example, a variable epitope library and compositions thereof, comprises or encodes peptides in which the amino acid residues are represented by “P1P2P3 . . . Pn”, where the numbers represent positions (P) of the various wild type amino acids, and where “n” represents the total polypeptide length and the position of the last amino acid. In various embodiments disclosed herein, at least one amino acid and as many as 90% of wild type amino acid residues can be randomly replaced by any of the 20 naturally occurring amino acid residues. Also, as one of skill in the art would readily recognize based, a variable epitope library includes polypeptides that are not yet known or identified, which enables a variable epitope library to induce a broad range of protective immune responses when introduced to a subject before one or more mutated epitopes (before infection) emerges or when the amount of one or more mutated epitopes is low (early stages of infection and/or disease progression).

In alternative embodiments, a variable epitope library composition can contain nucleic acid sequence molecules comprising from about 20 to about 200 subject nucleotides that encode the variable epitope polypeptides. In other embodiments, a variable epitope library composition can contain one or more polypeptide molecules where from about 10% to about 50% of the total amino acids of the one or more polypeptide molecules are variable amino acids (replaced by any of the 20 naturally occurring amino acid residues or a variant of a naturally occurring amino acid). In other embodiments, a variable epitope library composition can contain one or more polypeptides in which from about 20% to about 50% of the total amino acids of the one or more peptides are variable amino acids. In certain embodiments, a variable epitope library composition can contain one or more polypeptides in which from about 30% to about 50% of the total amino acids of the one or more peptides are variable amino acids. In yet other embodiments, a variable epitope library composition can contain one or more polypeptides in which from about 20% to about 40% of the total amino acids of the one or more peptides are variable amino acids.

The following examples are representative of a composition according to the invention. A variable epitope library composition as disclosed herein can be composed of a plurality of peptides, e.g., decapeptides, where a decapeptide:

P1-P2-P3-P4-P5-P6-P7-P8-P9-P10, where “P” refers to amino acid position and the number following P position of an amino acid in the decapeptide. According to the invention, in such a peptide of 10 amino acids in length, there can be up to 50% of its residue positions varying (designated as X below) and thus referred to as variant residues. The above decapeptide in which 50% of its residues are invariant (P below) and 50% are variant amino acid positions (X below, with the number following X referring to the position in the decapeptide) can be represented as:

P1-X2-P3-X4-P5-X6-P7-X8-P9-X10, where X can be any of the 20 naturally occurring amino acids or non-naturally occurring amino acids, and where P is an amino acid that is the same amino acid as that of the wild type epitope at that position.

According to the invention, another version of VEL composition based on the same decapeptide may be constructed by replacing wild type amino acid residues by X residues at odd positions and leaving this time wild type residues at even positions. This is represented as follows:

X1-P2-X3-P4-X5-P6-X7-P8-X9-P10.

While in these two particular decapeptide-based VELs composition each individual library member has 50% of wild type (invariant) and 50% varying amino acid positions, a composition according to the invention may be based on SARS-CoV-2 epitope in which only a single position of the epitope is varied (variant position). A composition according to the invention also may be based on a SARS-CoV-2 epitope in which as many as 90% of the epitope positions are variant residues, leaving 10% of the positions as invariant. Where a SARS-CoV-2 epitope contains an even number of amino acid positions, one can set forth invariant/variant amino acid positions of the epitope in terms of a ratio (e.g., a 1:1 ratio of invariant/variant positions characterizes the decapeptides set forth above).

VEL compositions comprise a combinatorial peptide library comprising individual peptides as described herein. The field of combinatorial peptide chemistry has emerged as a powerful tool in the study of many biological systems. In certain immunobiological applications, peptide libraries have proven monumental in the definition of MHC anchor residues, in lymphocyte epitope mapping, and in the development of peptide vaccines. Peptides identified from such libraries, when presented in a chemical microarray format, may prove useful in immunodiagnostics. Such peptide libraries offer a high-throughput approach to study limitless biological targets. Peptides discovered from such studies may be therapeutically and diagnostically useful agents.

Alternatively, multiple epitopes may be incorporated into the same molecule by recombinant technology well known in the art (Mateo et al., 1999; Astori and Krachenbuhl, 1996).

In one embodiment, a VEL composition contains variants of a CTL epitope, preferably a dominant CTL epitope, where 30-50% of amino acids at positions within the epitope other than the anchor positions are replaced by one of the 20 natural amino acids or variants thereof. A dominant CTL epitope is an epitope to which a functionally significant host immune response, e.g., an antibody response or a T-cell response, is made. Thus, with respect to a protective immune response against a pathogen, a dominant antigenic epitope is recognized by the host immune system result in protection from disease caused by the pathogen. An anchor position of the peptide aids in the quality of the binding interaction between the anchor residue of the peptide and the first pocket of the MHC class binding groove and is recognized as being a major determinant of overall binding affinity for the whole peptide.

Any of the known mutagenesis methods may be employed to generate the epitope variant peptides including cassette mutagenesis. These methods may be used to make amino acid modifications at desired positions of the peptide epitope. In one example, VEL compositions disclosed herein may be prepared by expression in a bacterial, viral, phage display, or eukaryotic expression system. In another example, the VEL compositions may be expressed and displayed on the surface of a recombinant bacteriophage, bacterium or yeast cell. The complexity of the library or vaccine composition can be up to about 20⁸ synthetic peptides.

A preferred method according to the invention refers to a randomly modified nucleic acid molecule coding for an epitope or mimotope, or a variant thereof which comprises at least one nucleotide repeating unit within non anchor positions having the sequence 5′-NNN-3′, 5′-NNS-3′, 5′-NNN-3′, 5′-NNB-3′ or 5′-NNK-3′. In some embodiments the modified nucleic acid comprises nucleotide codons selected from the group of TMT, WMT, BMT, RMC, RMG, MRT, SRC, KMT, RST, YMT, MKC, RSA, RRC, NNK, NNN, NNS or any combination thereof (the coding is according to IUPAC).

Assays

As discussed above, substructures of antigens are generally referred to as “epitopes” (e.g. B-cell epitopes, T-cell epitopes), as long as they are immunologically relevant, i.e. are also recognizable by antibodies and/or T cell receptors. T cell epitopes are generally linear epitopes of antigens and can be classified based on their binding affinity for mouse major histocompatibility complex (MHC) alleles. MHC class I T cell epitopes are generally about 9 amino acids long, ranging from 8-10 amino acids, while MHC class II T cell epitope are generally longer (about 15 amino acids long) and have less size constraints.

As is well-known in the art, there are a variety of screening technologies that may be used for the identification and isolation of desired peptide proteins capable of associating with MHC molecules, to form a complex recognized by a T cell receptor, with certain binding characteristics and affinities, including, for example, display technologies such as phage display, ribosome display, cell surface display, and the like, as described below. Methods for production and screening of variants are well-known in the art. Peripheral blood mononuclear cells (PBMCs) can be used as the source of CTL precursors. Those peptides able to induce in vitro proliferation of host peripheral blood mononuclear cells identify epitopes and/or mimotopes and/or variants thereof, to serve as a molecular component of personalized vaccines against cancer, infectious agents, such as the SARS-CoV-2 virus, or other diseases in an individual host both in prophylactic and therapeutic settings.

Antigen presenting cells are incubated with peptide, after which the peptide-loaded antigen-presenting cells are then incubated with the responder cell population under optimized culture conditions. Positive CTL activation can be determined by assaying the culture for the presence of CTLs that lyse radio-labeled target cells, either specific peptide-pulsed targets or target cells that express endogenously processed antigen from which the specific peptide was derived. Alternatively, the presence of epitope-specific CTLs can be determined interferon secretion assays or ELISPOT assays, including Interferon gamma (IFNy) in situ ELISA.

In accordance with these embodiments, the composition of an epitope of a pathogen-specific nucleic acid or polypeptide disclosed herein may be selected from one or more epitopes of SARS-CoV-2.

Epitopes are present in nature, and can be isolated, purified or otherwise prepared or derived by humans. For example, epitopes can be prepared by isolation from a natural source, or they can be synthesized in accordance with standard protocols in the art. Variants of synthetic epitopes can comprise artificial amino acid residues, such as D isomers of naturally-occurring L amino acid residues or non-naturally-occurring amino acid residues such as cyclohexylalanine. Throughout this disclosure, epitopes may be referred to in some cases as peptides or peptide epitopes. T cell epitopes are generally linear epitopes of antigens and can be classified based on their binding affinity for mouse major histocompatibility complex (MHC) alleles. MHC class I T cell epitopes are generally about 9 amino acids long, ranging from 8-12 amino acids, while MHC class II T cell epitope are generally longer (about 15-22 amino acids long) and have less size constraints.

T cell epitopes of antigens associated with a particular pathogen such as SARS-CoV-2, can be preliminarily identified using prediction tools known in the art, such as those located at the Immune Epitope Database and Analysis Resource (IEDB-AR), a database of experimentally characterized immune epitopes (B and T cell epitopes) for humans, nonhuman primates, rodents, and other animal species (http://tools.immuneepitope.org/analyze/html/mhc_binding.html).

Programs are available which provide high-accuracy predictions for peptide binding to human leucocyte antigen (HLA)-A or -B molecule with known protein sequence, as well as to MHC molecules from several non-human primates, mouse strains and other mammals). Lundegaard et al., Immunology 2010 July; 130(3): 309-318.

“T cell Repertoire”, on a nuclear level means a set of distinct recombined nucleotide sequences that encode T cell receptors (TCRs), or fragments thereof, in a population of T-lymphocytes of a subject, wherein the nucleotide sequences of the set have a one-to-one correspondence with distinct T-lymphocytes or their clonal subpopulations for substantially all of the T-lymphocytes of the population. In one aspect, a population of lymphocytes from which a repertoire is determined is taken from one or more tissue samples, such as peripheral blood monocytes (PBMC)s.

VEL libraries and VEL vaccine compositions disclosed herein can be administered to a subject prophylactically or therapeutically to treat, prevent, and/or reduce the risk of developing various diseases from various pathogens, such as SARS-CoV-2. Methods disclosed herein can include methods of preventing and/or treating SARS-CoV-2, in a subject including administering peptide epitopes, variants thereof, which associate with a subject's MHC class I molecules and which are identified from VEL libraries based on the peptide's in vitro interaction, or lack thereof, with the unique subset of a subject's T cell repertoire, based on a lymphoproliferation assay of the subject's PBMCs.

In one embodiment, T cell proliferation assays involve the analysis of PBMCs from healthy subjects and patients (for patients infected with SARS-CoV-2) in both total cell proliferation assays by fluorescence-activated cell sorting (FACS) and cell phenotyping assays (for example, as described in NoeDominguez-Romero et al., (2014) Human Vaccines & Immunotherapeutics, 10(11):3201-3213, incorporated herein by reference, with mice spleen cells). In one embodiment, cell phenotyping involves determination of the subpopulations of proliferating T cells (e.g., CD4+ and CD8+ cells) using flow cytometry and intracellular cytokine staining (ICS) for IFIN-y assays. For example, PBMCs are analyzed by FACS either after 6 hours of stimulation or upon 3 days of incubation with phage-displayed variant epitopes showing superior antigenic properties in a cell proliferation assay described above compared with corresponding wild-type epitope and a non-related epitope. Also, a standard ELISPOT assay could be used as described (Gallou C. et al, Oncotarget. 2016 Aug. 5. doi: 10.18632/oncotarget.11086. [Epub ahead of print] hereby incorporated by reference herein in its entirety) or as described in Current Protocols in Immunology (Greene Pub. Associates, U.S., hereby incorporated by reference herein in its entirety) or any other Immunological Protocols known to one of skill. In one embodiment, randomly selected phage-displayed variant epitopes/mimotopes can be used as antigens (10⁷-10′ particles/well) or synthetic peptides (10⁻⁶M) randomly (in silico) selected from epitope VEL libraries described herein. In one example, 1000 randomly selected phage phage-displayed variant epitopes from an epitope derived VEL library bearing a complexity of 8000 subject members are screened in assays, including a cell proliferation assay of PBMCs from a patient. However, the number of phage/peptides randomly selected phage can vary from 1 or up to 5, or up to 10, 20, 50, 100, 200, 250, 400, 500, 750, 1000, 2000, 4,000, or higher. Similarly, screening of libraries (phage or peptide or otherwise) in the methods disclosed herein can comprise random selection of individual library members or non-random selection of individual library members, and can include as few as one member, to as many as up to and including 10%, 20%, 30%, 40%, 50% 60%, 70%, 80%, 90% to 100% of the individual library members.

Genetic variability of antigens of many pathogens such as SARS-CoV-2, can result in the selection of mutated epitope variants in the patient which are able to escape control by immune responses. This can be a major obstacle to treatment strategies against infection by certain pathogens. Preferable embodiments herein relate to the characterization of peptides from variable epitope libraries, which are derived from SARS-CoV-2 pathogen antigens, preferably peptides able to bind MHC Class I molecules, with respect to their ability to interact with PBMC, especially CTLs, from a subject, in order to select peptides to administer to the subject which are effective to prevent or treat the SARS-CoV-2 associated disease or disorder afflicting the subject. Treatment of a SARS-CoV-2 disease or disorder afflicting the subject encompasses any amelioration of the disease or disorder, or symptoms thereof, whether temporary or permanent.

The complexities of VELs can range from a VEL composed of 20 epitope variants or mimotope variants, where only one wild-type amino acid residue is replaced in the epitope or mimotope by a random amino acid (e.g., 20 total peptides in the VEL), and up to about 20⁷ epitope variants, where several amino acid residues are mutated. In some embodiments, the complexities of VELs can range from about 20 different amino acids to about 20², or 20³ or 20⁴ different amino acids, depending on the number of variable amino acids, as one of skill in the art would recognize and understand based on the present disclosure and common knowledge. A VEL-based peptide can represent antigenic diversity observed during the course of SARS-CoV-2 associated disease or disorder, including resulting from an infection with a SARS-CoV-2, and/or subsequent infection with a different strain. Use of VEL immunogens as disclosed herein permits the generation of novel prophylactic and therapeutic vaccines and treatments capable of inducing a broad range of protective immune responses before the appearance of mutated epitopes (before pathogen infection) or when the amounts of mutated epitopes are low (early stages of pathogen infection and/or disease progression).

VELs are preferably generated based on a defined antigen of the SARS-CoV-2 pathogen or disease-related antigen-derived cytotoxic T lymphocyte (CTL). The epitopes are preferably derived from antigenically variable or relatively conserved regions of the protein antigen. Alternatively, VELs can be generated based on up to 50 amino acid long peptide regions of antigens containing clusters of epitopes. An individual VEL can contain: [1] a CTL epitope and variants of one CTL epitope; [2] variants of several different CTL epitopes; [3] any combination of [1] to [2]. In one embodiment a VEL is generated based on a CTL peptide epitope of 7-12 amino acids selected from a tumor antigen or from an antigenically variable or a relatively conserved region of a pathogen- or disease-related protein without a prior knowledge of the existence of epitopes in these peptide regions. Candidate CTL epitopes can be selected from scientific literature or from public databases. A VEL comprising a CTL epitopes and/or epitope variants thereof, in VELs are important since the escape from protective CTL responses is an important mechanism for immune evasion by SARS-CoV-2.

VEL—Nucleic Acid

VELs can take the form of DNA constructs, recombinant polypeptides or synthetic peptides and can be generated using standard molecular biology or peptide synthesis techniques, as discussed below. For example, to generate a DNA fragment encoding peptide variants of a particular epitope, a synthetic 4070 nucleotide (nt) long oligonucleotide (oligo) carrying one or more random amino acid-coding degenerate nucleotide triplet(s) may be designed and produced. The epitope-coding region of this oligo (oligol) may contain non-randomized 9-15 nucleotide segments at 5′ and 3′ flanking regions that may or may not encode natural epitope-flanking 3-5 amino acid residues. Then, 2 oligos that overlap at 5′ and 3′ flanking regions of oligol and carry nucleotide sequences recognized by hypothetical restriction enzymes A and B, respectively, may be synthesized and after annealing reaction with oligol used in a PCR. This PCR amplification will result in mutated epitope library-encoding DNA fragments that after digestion with A and B restriction enzymes may be combined in a ligation reaction with corresponding bacterial, viral or eukaryotic cloning/expression vector DNA digested with the same enzymes. Ligation mixtures can be used to transform bacterial cells to generate the VEL and then expressed as a plasmid DNA construct, in a mammalian virus or as a recombinant polypeptide. This DNA can also be cloned in bacteriophage, bacterial or yeast display vectors, allowing the generation of recombinant microorganisms.

In a similar manner, DNA fragments bearing 20-200 individual nucleotides can encode various combinations of different mutated epitope variants or mimotope variants. These nucleic acid molecules can be created using sets of long overlapping oligos and a pair of oligos carrying restriction enzyme recognition sites and overlapping with adjacent epitope-coding oligos at 5′ and 3′flanking regions. These oligos can be combined, annealed and used in a PCR assembly and amplification reactions. The resulting DNAs may be similarly cloned in vectors, e.g., mammalian virus vectors, and expressed as recombinant peptides or by recombinant microorganisms. The peptides may be used individually in immunotherapy or may be combined and used as a mixture of peptides.

In one example, synthetic peptide VELs varying in length from 7 to 12 amino acid residues may be generated by solid phase Fmoc peptide synthesis technique where in a coupling step equimolar mixtures of all proteogenic amino acid residues may be used to obtain randomized amino acid positions. This technique permits the introduction of one or more randomized sequence positions in selected epitope sequences and the generation of VELs with complexities of up to 10⁹, though preferably ranging from about 100 to 1000.

Peptide variants of an epitope based on VELs can be assessed and selected based on their interaction with a subject's PBMC, which are a source of CTLs. Thus, selected peptide variants of an epitope or a mimotope can be useful for inducing immune responses, especially CTL response against tumors and pathogens with antigenic variability as well as may be effective in modulating allergy, inflammatory and autoimmune diseases.

Pharmaceutical Compositions

In one embodiment, pharmaceutical compositions containing one or more VEL derived, selected peptide variants of a CTL epitope or a mimotope may be formulated with a pharmaceutically acceptable carrier, excipient and/or adjuvant, and administered to the subject, such as a non-human animal or a human patient. These pharmaceutical compositions can be administered to a subject, such as a human, therapeutically or prophylactically at dosages ranging from about 100 μg to about 1 mg of isolated peptides. Compositions containing VELs including nucleic acid sequences of the above peptides can be administered to a subject, such as a human, therapeutically or prophylactically at dosages ranging from about 1×10¹⁰ to about 5×10⁵ CFU of bacteriophage particles. In some embodiments, these pharmaceutical peptide or nucleic acid compositions administered to a human subject can reduce onset of a COVID-19 associated disease or disorder and/or can treat a COVID-19 associated disease or disorder already existing in the human subject. Other approaches for the construction of VELs, expression and/or display vectors, optimum pharmaceutical composition, routes for peptide or nucleic acid delivery and dosing regimens capable of inducing prophylactic and/or therapeutic benefits may be determined by one skilled in the art based on the present disclosure. For example, compositions containing these pharmaceutical peptide or nucleic acid compositions can be administered to a subject as a single dose application, as well as a multiple dose (e.g., booster) application. Multiple dose applications can include, for example, administering from about 1 to about 25 total dose applications, with each dose application administered at one or more dosing intervals that can range from about 7 days to about 14 days (e.g., weekly). In some embodiments, dosing intervals can be administered daily, two times daily, twice weekly, weekly, monthly, bi-monthly, annually, or bi-annually, depending on the particular needs of the subject and the characteristics of the condition being treated or prevented (or reducing the risk of getting the condition), as would be appreciated by one of skill in the art based on the present disclosure.

Amino Acids

The skilled artisan will realize that in alternative embodiments, less than the 20 naturally occurring amino acids may be used in a randomization process. For example, certain residues that are known to be disruptive to protein or peptide secondary structure, such as proline residues, may be less preferred for the randomization process. VELs may be generated with the 20 naturally occurring amino acid residues or with some subset or variants of the 20 naturally occurring amino acid residues. In various embodiments, in addition to or in place of the 20 naturally occurring amino acid residues, the VELs may contain at least one modified amino acid, as indicated in the below table 1.

[symbol]

Modified amino acid residues

| Abbr. | Amino Acid |
|---|---|
| Aad | 2-Aminoadipic acid |
| Baad | 3-Aminoadipic acid |
| Bala | β-alanine, |
| | β-Amino-propionic acid |
| Abu | 2-Aminobutyric acid |
| 4Abu | 4-Aminobutyric acid, |
| | piperidinic acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| AHyl | allo-Hydroxylysine |
| 3Hyp | 3-Hydroxyproline |
| 4Hyp | 4-Hydroxyproline |
| Ide | Isodesmosine |
| AIle | allo-Isoleucine |
| MeGly | N-Methylglycine, |
| | sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

[symbol]

indicates data missing or illegible when filed

Combinatorial Libraries

Combinatorial libraries of such compounds or of such targets can be categorized into several categories. The first category relates to the matrix or platform on which the library is displayed and/or constructed. For example, combinatorial libraries can be provided (i) on a surface of a chemical solid support, such as micro-particles, beads or a flat platform; (ii) displayed by a biological source (e.g., bacteria or phage); and (iii) contained within a solution. In addition, three dimensional structures of various computer generated combinatorial molecules can be screened via computational methods.

Another category of combinatorial libraries relates to the method by which the compounds or targets are synthesized, such synthesis is typically effected by: (i) in situ chemical synthesis; (ii) in vivo synthesis via molecular cloning; (iii) in vitro biosynthesis by purified enzymes or extracts from microorganisms; and (iv) in silico by dedicated computer algorithms.

Combinatorial libraries indicated by any of the above synthesis methods can be further characterized by: (i) split or parallel modes of synthesis; (ii) molecules size and complexity; (iii) technology of screening; and (iv) rank of automation in preparation/screening.

VEL Expression

In certain embodiments, it may be preferred to make and use an expression vector that encodes and expresses a particular VEL. Gene sequences encoding various polypeptides or peptides may be obtained from GenBank and other standard sources, as disclosed above. Expression vectors containing genes encoding a variety of known proteins may be obtained from standard sources, such as the American Type Culture Collection (Manassas, Va.). For relatively short VELs, it is within the skill in the art to design synthetic DNA sequences encoding a specified amino acid sequence, using a standard codon table, as discussed above. Genes may be optimized for expression in a particular species of host cell by utilizing well-known codon frequency tables for the desired species.

Regardless of the source, a coding DNA sequence of interest can be inserted into an appropriate expression system. The DNA can be expressed in any number of different recombinant DNA expression systems to generate large amounts of the polypeptide product, which can then be purified and used in various embodiments of the present disclosure.

Examples of expression systems known to the skilled practitioner in the art include bacteria such as E. coli, yeast such as Pichia pastoris, baculovirus, and mammalian expression systems such as in Cos or CHO cells. Expression is not limited to single cells but may also include protein production in genetically engineered transgenic animals, such as mice, rats, cows or goats.

The nucleic acid encoding a peptide may be inserted into an expression vector by standard subcloning techniques. An E. coli expression vector may be used which produces the recombinant polypeptide as a fusion protein, allowing rapid affinity purification of the peptide. Examples of such fusion protein expression systems are the glutathione S-transferase system (Pharmacia, Piscataway, N.J.), the maltose binding protein system (NEB, Beverley, Mass.), the FLAG system (IBI, New Haven, Conn.), and the 6×His system (Qiagen, Chatsworth, Calif.).

Some of these systems produce recombinant polypeptides bearing only a small number of additional amino acids, which are unlikely to affect the activity or binding properties of the recombinant polypeptide. For example, both the FLAG system and the 6×His system add only short sequences, both of which have no adverse effect on folding of the polypeptide to its native conformation. Other fusion systems are designed to produce fusions wherein the fusion partner is easily excised from the desired peptide. In one embodiment, the fusion partner is linked to the recombinant peptide by a peptide sequence containing a specific recognition sequence for a protease. Examples of suitable sequences are those recognized by the Tobacco Etch Virus protease (Life Technologies, Gaithersburg, Md.) or Factor Xa (New England Biolabs, Beverley, Mass.). The expression system used may also be one driven by the baculovirus polyhedron promoter. The gene encoding the polypeptide may be manipulated by standard techniques in order to facilitate cloning into the baculovirus vector. One baculovirus vector is the pBlueBac vector (Invitrogen, Sorrento, Calif.). The vector carrying the gene for the polypeptide is transfected into Spodoptera frugiperda (Sf9) cells by standard protocols, and the cells are cultured and processed to produce the recombinant protein.

In one embodiment expression of a recombinant encoded peptide comprises preparation of an expression vector that comprises one of the isolated nucleic acids under the control of, or operatively linked to, one or more promoters. To bring a coding sequence “under the control of” a promoter, the 5′ end of the transcription initiation site of the transcriptional reading frame is positioned generally from about 1 to about 50 nucleotides “downstream” (3′) of the chosen promoter. The “upstream” promoter stimulates transcription of the DNA and promotes expression of the encoded recombinant protein.

Many standard techniques are available to construct expression vectors containing the appropriate nucleic acids and transcriptional/translational control sequences in order to achieve peptide expression in a variety of host-expression systems. Cell types available for expression include, but are not limited to, bacteria, such as E. coli and B. subtilis transformed with recombinant bacteriophage DNA, plasmid DNA or cosmid DNA expression vectors. Non-limiting examples of prokaryotic hosts include E. coli strain RR1, E. coli LE392, E. coli B, E. coli X 1776 (ATCC No. 31537) as well as E. coli W3110 (F-, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is often transformed using pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which may be used by the microbial organism for expression of its own proteins.

In addition, phage vectors containing replicon and control sequences that are compatible with the host microorganism may be used as transforming vectors in connection with these hosts. For example, the phage lambda GEMTM-11 may be utilized in making a recombinant phage vector which may be used to transform host cells, such as E. coli LE392.

Further useful vectors include pIN vectors and pGEX vectors, for use in generating glutathione S transferase (GST) soluble fusion proteins for later purification and separation or cleavage. Other suitable fusion proteins are those with 13-galactosidase, ubiquitin, or the like. Preferable promoters for use in recombinant DNA construction include the 13-lactamase (penicillinase), lactose and tryptophan (trp) promoter systems. However, other microbial promoters have been discovered and utilized, and details concerning their nucleotide sequences have been published, enabling those of skill in the art to ligate them functionally with plasmid vectors.

For expression in Saccharomyces, the plasmid YRp7, for example, is commonly used. This plasmid already contains the trpl gene which provides a selection marker for a mutant strain of yeast lacking the ability to grow in tryptophan, for example ATCC No. 44076 or PEP4-1. The presence of the trp/lesion as a characteristic of the yeast host cell genome then provides an effective environment for detecting transformation by growth in the absence of tryptophan.

Suitable promotor sequences in yeast vectors include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other suitable promoters, which have the additional advantage of transcription controlled by growth conditions, include the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization.

In addition to micro-organisms, cultures of cells derived from multicellular organisms may also be used as hosts. In principle, any such cell culture is workable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing one or more coding sequences.

In a preferable insect system, Autographa californica nuclear polyhidrosis virus (AcNPV) is used as a vector to express foreign genes. The virus grows in Spodoptera frugiperda cells. The isolated nucleic acid coding peptide sequences are cloned into non-essential regions (e.g., polyhedrin gene) of the virus and placed under control of an AcNPV promoter (e.g., polyhedrin promoter). Successful insertion of the coding sequences results in the inactivation of the polyhedrin gene and production of non-occluded recombinant virus (e.g., virus lacking the proteinaceous coat coded for by the polyhedrin gene). These recombinant viruses are then used to infect Spodoptera frugiperda cells in which the inserted nucleic acid coding the peptide sequences is expressed.

Examples of preferable mammalian host cell lines are VERO and HeLa cells, Chinese hamster ovary (CHO) cell lines, W138, BHK, COS-7, 293, HepG2, 3T3, RIN and MDCK cell lines. In addition, a host cell strain may be chosen that modulates the expression of the inserted peptide encoding sequences or modifies and processes the peptide product in the specific fashion desired.

Different host cells have characteristic and specific mechanisms for the post-translational processing and modification of proteins. Appropriate cells lines or host systems may be chosen to ensure the correct modification and processing of the foreign peptide expressed. Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter) as known in the art.

A number of viral based expression systems may be utilized, for example, commonly used promoters are derived from polyoma, Adenovirus 2, and most frequently Simian Virus 40 (SV40). The early and late promoters of SV40 virus are useful because both are obtained easily from the virus as a fragment which also contains the SV40 viral origin of replication. Smaller or larger SV40 fragments may also be used, provided there is included the approximately 250 bp sequence extending from the Hind III site toward the Bgl I site located in the viral origin of replication.

In one example where an adenovirus is used as an expression vector, the peptide coding sequences may be ligated to an adenovirus transcription/translation control complex (e.g., the late promoter and tripartite leader sequence). This chimeric gene may then be inserted in the adenovirus genome by in vitro or in vivo recombination. Insertion in a non-essential region of the viral genome (e.g., region E1 or E3) will result in a recombinant virus that is viable and capable of expressing the peptides in infected hosts.

Specific initiation signals known in the art may also be required for efficient translation of the claimed isolated nucleic acid encoding the peptide sequences. One of ordinary skill in the art would readily be capable of determining this and providing the necessary signals.

A number of selection systems may be used, including but not limited to, the herpes simplex virus thymidine kinase, hypoxanthine-guanine phosphoribosyltransferase and adenine phosphoribosyltransferase genes, in tk⁻, hgprt⁻ or aprt⁻ cells, respectively. Also, antimetabolite resistance may be used as the basis of selection for dihydrofolate reductase (DHFR), which confers resistance to methotrexate; xanthineguanine phosphoribosyl transferase (gpt), which confers resistance to mycophenolic acid; neomycin (neo), that confers resistance to the aminoglycoside G-418; and hygro, which confers resistance to hygromycin. These and other selection genes may be obtained in vectors from, for example, ATCC or may be purchased from a number of commercial sources known in the art (e.g., Stratagene, La Jolla, Calif.; Promega, Madison, Wis.).

Where substitutions of a pathogen- or disease-related epitope or mimotope thereof are desired, the nucleic acid sequences encoding the substitutions may be manipulated by well-known techniques, such as site-directed mutagenesis or by chemical synthesis of short oligonucleotides followed by restriction endonuclease digestion and insertion into a vector, by PCR based incorporation methods, or any similar method known in the art.

Protein Purification

In certain embodiments the peptide(s) may be isolated or purified. Protein purification techniques are well known to those of skill in the art. These techniques involve, at one level, the homogenization and crude fractionation of the cells to peptide and non-peptide fractions. The peptide(s) of interest may be further purified using chromatographic and electrophoretic techniques to achieve partial or complete purification (or purification to homogeneity). Analytical methods well suited to the preparation of a pure peptide are ion-exchange chromatography, gel exclusion chromatography, polyacrylamide gel electrophoresis, affinity chromatography, immunoaffinity chromatography and isoelectric focusing. An efficient method of purifying peptides is fast performance liquid chromatography (FPLC) or even HPLC.

A purified peptide is intended to refer to a composition, isolatable from other components, wherein the peptide is purified to any degree. An isolated or purified polypeptide or peptide, therefore, also refers to a polypeptide or peptide free from the environment from which it originated. Generally, “purified” will refer to a peptide composition that has been subjected to fractionation to remove various other components. Where the term “substantially purified” is used, this designation will refer to a composition in which the peptide forms the major component of the composition, such as constituting about 50%, about 60%, about 70%, about 80%, about 90%, about 95%, or more of the peptides in the composition. Various methods for quantifying the degree of purification of the peptide are known to those of skill in the art in light of the present disclosure.

Various techniques suitable for use in peptide purification are contemplated herein and are well known. There is no general requirement that the peptide always be provided in their most purified state. Indeed, it is contemplated that less substantially purified products will have utility in certain embodiments. In another embodiment, affinity chromatography may be required and any means known in the art is contemplated herein.

Formulations and Routes for Administration to Subjects

Where clinical applications are contemplated, it will be necessary to prepare pharmaceutical compositions (e.g., VEL peptide compositions) in a form appropriate for the intended application. Generally, this will entail preparing compositions that are essentially free of impurities that could be harmful to human or animal subjects.

Preferably, the peptide compositions comprise salts and buffers to render the peptides stable and allow for interaction with target cells. Aqueous compositions may comprise an effective amount of peptide dissolved or dispersed in a pharmaceutically acceptable carrier or aqueous medium. Such compositions also are referred to as innocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the polypeptides of the present disclosure, its use in therapeutic compositions is contemplated. Supplementary active ingredients also can be incorporated into the compositions.

The active peptide compositions instantly disclosed include classic pharmaceutical preparations. Administration of these compositions according to the present disclosure will be via any common route. This includes oral, nasal, buccal, rectal, vaginal, topical, orthotropic, intradermal, subcutaneous, intramuscular, intraperitoneal, intraarterial or intravenous injection. Such compositions normally would be administered as pharmaceutically acceptable compositions, as described above.

The active peptide compounds also may be administered parenterally or intraperitoneally. Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent needed for easy application via syringe. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In certain examples, it will be preferable to include isotonic agents, for example, sugars or sodium chloride. Prolonged absorption of the injectable compositions can be brought about by the use in the compositions of agents delaying absorption, for example, aluminum monostearate and gelatin. Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. Regarding sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The VELs and VEL peptide compositions of the present disclosure may also be used in conjunction with targeted therapies, including but not limited to, therapies designed to target pathogens including the SARS-CoV-2 pathogen, and the cells infected with the pathogen. Many different targeted therapies are being explored for use in treatment of infection by the SARS-CoV-2 pathogen. For example, these therapies can include hormone therapies, signal transduction inhibitors, gene expression modulator, apoptosis inducer, angiogenesis inhibitor, immunotherapies, and toxin delivery molecules.

Cell Proliferation Assays

Lymphocyte proliferation assay' comprises isolating peripheral blood mononuclear cells (PBMCs), placing 100,000 of the cells in each well of a 96-well plate with or without various stimuli, and allowing the cells to proliferate for six days at 37° C. in a CO₂ incubator. The amount of proliferation is detected on the sixth day by adding radioactive ³H (tritiated) thymidine for six hours, which is incorporated into the newly synthesized DNA of the dividing cells. The amount of radioactivity incorporated into DNA in each well is measured in a scintillation counter and is proportional to the number of proliferating cells, which in turn is a function of the number of lymphocytes that were stimulated by a given antigen to enter the proliferative response. The readout is counts per minute (cpm) per well.

Detailed Lymphocyte Proliferation Assay

Briefly, 10 ml of heparinized venous blood was drawn from each study subject. For WB assay, 1:5 and 1:10 dilutions were made with sterile RPMI 1640 medium (Sigma Chemical Company, MO, USA), supplemented with penicillin (100 !Wm!), streptomycin (0.1 mg/ml), L-glutamine (0.29 gm/l) and amphotericin B (5 mg/ml) and was seeded in 96-well flat bottom plates at 200 μl/well.

PBMC were isolated by Ficoll-Hypaque density centrifugation. A total of 2×10⁵ cells/well were cultivated in complete culture medium, supplemented with 10% Human AB serum. Cultures were stimulated either with candidate peptide (5 μg/nil), or PHA (5 μg/ml) as a positive control or PPD (5 μg/ml). Cells cultured under similar conditions without any stimulation served as the negative control. The cultures were set up in triplicates and incubated for 6 days at 37° C. in 5% CO₂ atmosphere. Sixteen hours before termination of cultures, 1 of tritiated (³H) thymidine (Board of Radiation and Isotope Technology, MA, USA) was added to each well. The cells were then harvested onto glass fiber filters on a cell harvester and allowed to dry overnight. 2 ml of scintillation fluid (0.05 mg/ml POPOP and 4 mg/ml PPO in lit. of toluene) was added to each tube containing the dried filter discs and counted by using a liquid scintillation beta counter.

The proliferation was measured as uptake of tritiated thymidine by cells and expressed as stimulation index (SI) which was calculated as Stimulation Index=mean counts per minute with peptide/mean counts per minute without peptide.

Interferon-y Measurement

For quantification of IFN-γ, in all 1:5 and 1:10 diluted blood and PBMC cell-free culture supernatants from lymphocyte proliferation assay were harvested after 6 days of in vitro stimulation with or without antigen stimuli and stored at −80° C. until assayed. IFN-γ production was determined by standard ELISA technique using commercially available BD opt-EIA Kit (BD Biosciences, Franklin Lakes, N.J., USA) as per the manufacturer's instructions.

Any part of this disclosure may be read in combination with any other part of the disclosure, unless otherwise apparent from the context.

The compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The present invention is described in more detail in the following non limiting exemplifications.

### Working Example I

STEP I Construction of Variable Epitope Libraries (VELs)

Development of VEL vaccines are useful both in prophylactic and therapeutic settings.

Step A. Epitopes of SARS-CoV-2 which bind widely expressed MHC haplotypes were identified. The present vaccines were designed, taking into account the prevailing MHC haplotypes of the Chinese population (see the attached FIG. 1 with the map), which also includes the Hubei Province.

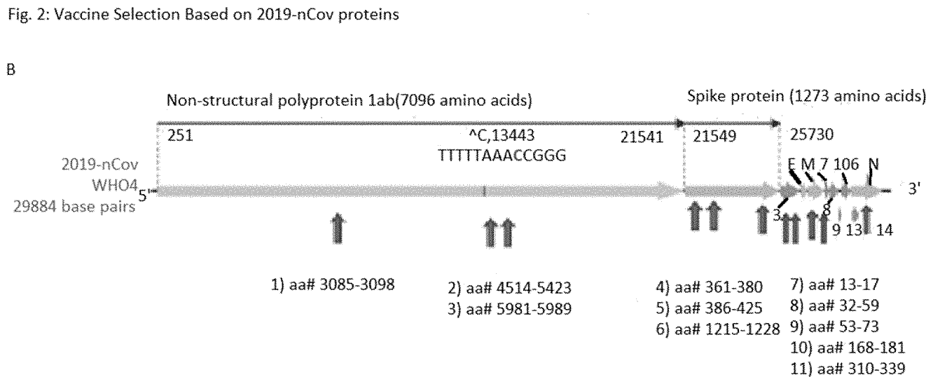

Step B. Identify multi-epitope regions from reported sequences of the virus using in silico methods covering major protein sequences of SARS-CoV-2 (FIG. 2).

The sequences of vaccine immunogens are summarized in Table 2 below.

| | GenBank | | | |
|---|---|---|---|---|
| 2019-nCoV | accession | | | MHC class I |
| protein | identifier | | Vaccine Sequences | molecule |
| Env | QHD43418.1 | Wild Type | 13 IVNSYLLFLAFVVFLLVTLAILTAL 37 (SEQ ID | HLA-A*02:01 |
| | | (WT) | NO: 1) | |
| | | VEL | 13 IVNSVLXFLAFXVFLLVTLXILTAL 37 (SEQ ID | HLA-A*02:01 |
| | | Vaccine | NO: 2) | |
| | | CoVV1 | | |
| | | WT | 32 AILTALRLCAYCCNIVNVSLVKPSFYVY 59 (SEQ ID | HLA-A*11:01 |
| | | | NO: 3) | |
| | | VEL | 32 AILTXLRLCAYXCNIVXVSLVKPXFYVY 59 (SEQ ID | HLA-A*11:01 |
| | | Vaccine | NO: 4) | |
| | | CoVV2 | | |
| Membrane | QHD43419.1 | WT | 53 FLWLLWPVTLACFVLAAVYRI 73 (SEQ ID NO: 5) | HLA-A*02:01 |
| glyco- | | CoVV3 | 53 FLINXLICPVTLXCFVLXAVYRI 73 (SEQ ID NO: 6) | HLA-A*02:01 |
| protein | | WT | 169 TVATSRTLSYYKL 181 (SEQ ID NO: 7) | HLA-A*11:01 |
| | | CoVV4 | 169 TVXTSRXLSXYKL 181 (SEQ ID NO: 8) | HLA-A*11:01 |
| Nucleo- | QHD43423.2 | WT | 310 SASAFFGMSRIGMEVTPSGTWLTYTGAIKL 339 | HLA-A02:01/ |
| capsid | | | (SEQ ID NO: 9) | HLA-A*11:01 |
| | | CoVV5 | 310 SAXAFXGMSRXGMEVTPSGTWLTYXGXIKL 339 | HLA-A*02:01/ |
| | | | (SEQ ID NO: 10) | HLA-A*11:01 |
| ORF1ab | QHD43415.1 | WT | 4514 YTMADLVYAL 4523 (SEQ ID NO: 11) | HLA-A*02:01 |
| | | CoVV6 | 4514 YTXADXVXAL 4523 (SEQ ID NO: 12) | HLA-A*02:01 |
| | | WT | 5981 SMMGFKMNY 5989 (SEQ ID NO: 13) | HLA-A*11:01 |
| | | CoVV7 | 5981 SMXGXKXNY 5989 (SEQ ID NO: 14) | HLA-A*11:01 |
| | | WT | 3085 FLMSFTVLCLTPVY 3098 (SEQ ID NO: 15) | HLA-A*02:01 |
| | | CoVV8 | 3085 FLMXFXVLCXTPVY 3098 (SEQ ID NO: 16) | HLA-A*02:01 |
| Spike | QHD43416.1 | WT | 386 | HLA-A*02:01 |
| Protein | | | KLNDLCFTNVYADSFVIRGDEVRQIAPGQTGKIADYN | |
| | | | YKL 425 (SEQ ID NO: 17) | |
| | | CoVV9 | 386 | HLA-A*02:01 |
| | | | KLNDLXFXNVYADSFVIRGDEXRQIAPGQTGKIADXN | |
| | | | XKL 425 (SEQ ID NO: 18) | |
| | | WT | 1215 YIWLGFIAGLIAIV 1228 (SEQ ID NO: 19) | HLA-A*02:01 |
| | | CoVV10 | 1215 YIWLXFIXGXIAIV 1228 (SEQ ID NO: 20) | HLA-A*02:01 |
| | | WT | 361 CVADYSVLYNSASFSTFKCY 380 (SEQ ID | HLA-A*11:01 |
| | | | NO: 21) | |
| | | CoVV11 | 361 CVADXSXLYNSASFSTXKCY 380 (SEQ ID | HLA-A*11:01 |
| | | | NO: 22) | |
| | | WT | 464 FERDISTEIYQAGSTPCNGVEGFNCYFPLQS 494 | HLA-A*02:01 |
| | | | (SEQ ID NO: 23) | |
| | | COV12 | 464 FERDISTEXYQXGXTPCNGXEXFNCYFPLQS 494 | HLA-A*02:01 |
| | | | (SEQ ID NO: 24) | |

X = any out of 20 proteinogenic amino acids

For Example:

(CoVV1) is a SARS-CoV-2 antigen listed in the above Table. It has a sequence of IVNSVLLFLAFVVFLLVTLAILTAL (SEQ ID NO:1). Phage display VELs and synthetic peptide VELs are generated based on the CoVV1-derived HLA-A*02:01 CTL IVNSVLXFLAFXVFLLVTLXILTAL, (SEQ ID NO:2), where X is any of the 20 naturally occurring amino acids or variants thereof.

VELs are generated using the recombinant M13 phage display system. To generate the VELs, molecular biology procedures are carried out using standard protocols, including the use of restriction enzymes, Taq DNA polymerase, DNA isolation/purification kits, T4 DNA ligase and M13K07 helper phages.

In order to express the CoVV1-derived wild-type CTL peptide epitope IVNSVLLFLAFVVFLLVTLAILTAL (SEQ ID NO:1) and epitope variant-bearing VELs on M13 phage surfaces as fusions with the major phage coat protein (cpVlll), corresponding DNA fragments are generated by PCR and cloned in a pG8SAET phagemid vector.

Correct sequences are verified using standard automated sequencers.

The resulting recombinant phage clone expressing the wild type epitope and the VEL phage library carrying epitope variants, are rescued/amplified using M13K07 helper phages by infection of E. coli TG1 cells and purified by double precipitation with polyethylene glycol (20% PEG/2.5 M NaCl). A number of phage clones are randomly selected from the VEL library, each expressing different epitope variants, and rescued/amplified from 0.8 mL of bacterial cultures using 96 well 1 mL round bottom blocks. The typical phage yields are 10¹⁰ to 10¹¹ colony forming units (CFU) per milliliter of culture medium. The DNA inserts of a number of phage clones from the VEL library are sequenced and the amino acid sequences of the peptides are deduced.

Thus, the DNA fragments corresponding to the wild type and variant epitopes, respectively, are amplified by PCR and are cloned into pG8SAET phagemid vector that allows the expression of epitopes at high copy numbers as peptides fused to phage gpVlll. The amino acids at the MHC-binding anchor positions are maintained within the epitope, while mutations are introduced at positions responsible for interaction with the T cell receptor (TCR). As each variant epitope has random amino acid substitutions (mutations) at 3 defined positions within the wild type epitope, the theoretical complexity of the library is 8×10³ individual members.

Cell Proliferation Assays

PBLs are obtained from a subject of interest having SARS-CoV-2, as well as from a healthy subject (or population of healthy subjects). The Subject peptides are then assayed for its interaction with PBMCs from said subject and from a healthy subject (or population of healthy subjects) based on an in vitro proliferation assay

In Vitro Stimulation:

The PBMCs are stimulated by culturing in a 96-well flat-bottom plate (2.5×10⁵ cells/well) with 10⁷-10¹⁰ phage particles/well corresponding to particular epitope variant for 72 hours at 37 C.° in CO₂ incubator. The gating strategy involves exclusion of doublets and dead cells; 10,000 lymphocytes (R1) are gated for a CD4+ versus CD8+ dot-plot graph to measure CD4+IFN-γ+, CD8+IFN-γ+ and proliferation percentages of CD4+CD8− and CD4-CD8+ cells.

Total cell proliferation and CD4+ and CD8+ T-cell responses are evaluated by using intracellular staining (ICS) for IFN-γ both ex vivo and in vitro by stimulating fresh lymphocytes for 6 hours or 72 hours, respectively. During the last 4 hours, 1 Monensin (2 uM) (a protein transport inhibitor) is added to the culture. The cells are stained with fluorescence-labeled monoclonal antibodies against CD4 and CD8 for 30 minutes at room temperature, are fixed with fixation buffer and, after washing, the cells are permeabilized with permeabilization wash buffer, and then are labeled for 30 minutes with anti-IFN-γ antibody in the dark. The cells are analyzed on FACSCalibur Cytometer using CellQuest software data acquisition and analysis program from BD Bioscience and operates in the Macintosh environment on the FACSCalibur cytometers; at least 10,000 events are collected.

Immunization of Subject with Positive Immunostimulatory Peptide

Immunostimulatory peptides showing the highest capacity to induce the proliferation of PBMCs obtained from a SARS-CoV-2 patient are determined by the in vitro data above and are used as an inoculum to administer to said subject for the prevention of SARS-CoV-2 infection. Alternatively, Immunostimulatory peptides as determined by the in vitro data above, are used as an inoculum to administer to said subject for treatment of SARS-CoV-2 in a subject suffering from a SARS-CoV-2 infection.

### REFERENCES

Pan, Q. et al. The distribution of HLA-A, -B, and -DRB1 alleles and haplotypes in inhabitants of Guizhou Province of China. J. Biomed. Res. 25, 328-334 (2011).
Li, X. F. et al. An analysis of HLA-A, -B, and -DRB1 allele and haplotype frequencies of 21,918 residents living in Liaoning, China. PLoS One 9, (2014).
Zhou, X. Y. et al. High-resolution analyses of human leukocyte antigens allele and haplotype frequencies based on 169, 995 volunteers from the China bone marrow donor registry program. PLoS One 10, 1-26 (2015).
Shao, L. N. et al. HLA-A, HLA-B, HLA-DRB1 allele and haplotype frequencies of 14 529 Chinese Han bone marrow donors living in Dalian, China. Int. J. Immunogenet. 43, 79-85 (2016).
MacKenzie and Smith Microbiol Aust. 2020 Mar. 17; MA20013. doi: 10.1071/MA20013 Zhu N., et al. N. Engl. J. Med. 2020; 382:727-733
Zhou P., et al. Nature. 2020; 579(7798)
Ren L. L., et al Chin Med J (Engl) 2020 Feb. 11
Lu R., et al. Lancet. 2020; 395:565-574; Wu, F., Zhao, S., Yu, B. et al. Nature 579, 265-269 World Health Organization Novel coronavirus (2019-nCoV). Situation Report 22. 11 Feb. 2020. https://www.who.int/docs/default-source/coronaviruse/situation-reports/20200211-sitrep-22-ncov.pdf?sfvrsn=fb6d49b1_2 (accessed 22 Feb. 2020)
Gorbalenva A. E., et al. (2020 Feb. 7) Severe Acute respiratory syndrome-related coronavirus: the species and its viruses—a statement of the Coronavirus Study Group. bioRiv, doi:10.1101/2020.02.07.937862
National Institutes of Health (NIH) https://www.niaid.nih.gov/diseases-conditions/coronaviruses) Claus Lundegaard et al., “Major histocompatibility complex class I binding predictions as a tool in epitope discovery” Immunology. 2010 July; 130(3): 309-318
Rock et al., “Proteases in MHC class I presentation and cross-presentation” J. Immunol. 2010 Jan. 1; 184(1): 9-1
Trevor Bedford, Fred Hutch expert, https://www.fredhutch.org/en/news/center-news/2020/03/tracking-covid-19-trevor-bedford.html
Mason D. (1998) Immunol Today 19:395-404
McMahan R. H., et al. J Clin Invest. 2006; 116:2543-2551
Wooldridge L., et al., J Biol Chem. 2011; 287:1168-1177
Parkhurst M. R., et al. J. Immunol., 1996; 157:2539-2548
Borbulevych O. Y., et al. J. Immunol. 2005; 174:4812-4820
Zaremba S., et al., Cancer Res. 1997; 57:4570-4577
Salazar E., et al., Int. J Cancer. 2000; 85:829-838
Gras et al., J. Exp. Med. (2010) Vol. 207 No. 7:1555-1567
Birnbaum M E., et al., (2014) Cell. 2014 May 22; 157(5):1073-87
Hoppes et al., (2014) J Immunol. 193(10):4803-13
Abdul-Alim C. S. et al., (2010) J Immunol., 1; 184(11):6514-21
Ekeruche-Makinde et al. (2010) J Biol Chem. 2012 October; 287(44):37269-81
Buhrman et al., (2013) J Biol Chem. 2013 Nov. 15; 288(46):33213-25
Kappler J. W. et al. (1987) Cell 49: 273-80
Hengartner H. et al., (1988) Nature 336: 388-90
Pircher H. et al., (1991) Nature 351: 482-526
Wooldridge L, et al. J Biol Chem. 2011; 287:1168-1177
Parkhurst M. R., et al. J Immunol. 1996; 157:2539-2548
Borbulevych O Y, et al. J Immunol. 2005; 174:4812-4820
Jonathan D. Buhrman and Jill E. Slansky, Immunol Res. 2013 March; 55: 34-47
McMahan R H, et al. J Clin Invest. 2006; 116:2543-2551
Zaremba S, et al. Cancer Res. 1997; 57:4570-4577
Salazar E, et al. Int J Cancer. 2000; 85:829-838
Kim J H, Davis W G, Sambhara S, Jacob J. Strategies to alleviate original antigenic sin responses to influenza viruses. Proc Natl Acad Sci USA 2012; 109(34):13751-6; PMID:22869731; http://dx.doi.org/10.1073/pnas.0912458109
Klenerman P, Zinkernagel R M. Original antigenic sin impairs cytotoxic T lymphocyte responses to viruses bearing variant epitopes. Nature 1998; 394:482-485; PMID:9697771
Bryant and Ploegh, Curr Opin Immunol 2004; 16:96-102

### Other Embodiments

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Other embodiments are within the following claims.

1. A method of treating and/or preventing disease resulting from viral infection in a subject by the virus SARS-CoV-2, the method comprising: administering a SARS-CoV-2 variable epitope library composition comprising one or more synthetic peptide(s), each said peptide comprising:
(i) an amino acid sequence identical to an epitope of a SARS-CoV-2 viral antigen or nucleic acid encoding said synthetic peptide(s), and/or
(ii) an amino acid sequence which differs from said epitope in at least one corresponding amino acid residue, wherein each said differing corresponding amino acid residue is a variable amino acid, or nucleic acid encoding said synthetic peptide(s),
wherein the length of each of said one or more peptide(s) ranges from 7 to 50 amino acids in length, and
wherein from about 1% to about 50% of the total amino acids of the one or more peptide(s) are variable amino acids, and
(iii) a pharmaceutically acceptable excipient,
thereby treating and/or preventing disease resulting from viral infection by SARS-CoV-2.

2. The method of claim 1, wherein the SARS-CoV-2 viral antigen comprises a CTL epitope, and wherein said CTL epitope comprises an amino acid sequence selected from the group consisting of: IVNSVLLFLAFVVFLLVTLAILTAL, AILTALRLCAYCCNIVNVSLVKPSFYVY, FLWLLWPVTLACFVLAAVYRI, TVATSRTLSYYKL, SASAFFGMSRIGMEVTPSGTWLTYTGAIKL, YTMADLVYAL, SMMGFKMNY, FLMSFTVLCLTPVY, KLNDLCFTNVYADSFVIRGDEVRQIAPGQTGKIADYNYKL, YIWLGFIAGLIAIV, CVADYSVLYNSASFSTFKCY and FERDISTEIYQAGSTPCNGVEGFNCYFPLQS.

3. The method of claim 2, wherein variants of said CTL epitope IVNSVLLFLAFVVFLLVTLAILTAL are IVNSVLXFLAFXVFLLVTLXILTAL, wherein variants of said CTL epitope AILTALRLCAYCCNIVNVSLVKPSFYVY are AILTXLRLCAYXCNIVXVSLVKPXFYVY; wherein variants of said CTL epitope FLWLLWPVTLACFVLAAVYRI are FLWXLXPVTLXCFVLXAVYRI, wherein variants of said CTL epitope TVATSRTLSYYKL are TVXTSRXLSXYKL, wherein variants of said CTL epitope SASAFFGMSRIGMEVTPSGTWLTYTGAIKL are SAXAFXGMSRXGMEVTPSGTWLTYXGXIKL, wherein variants of said CTL epitope YTMADLVYAL are YTXADXVXAL, wherein variants of said CTL epitope SMMGFKMNY are SMXGXKXNY, wherein variants of said CTL epitope FLMSFTVLCLTPVY are FLMXFXVLCXTPVY, wherein variants of said CTL epitope KLNDLCFTNVYADSFVIRGDEVRQIAPGQTGKIADYNYKL are KLNDLXFXNVYADSFVIRGDEXRQIAPGQTGKIADXNXKL, wherein variants of said CTL epitope YIWLGFIAGLIAIV are YIWLXFIXGXIAIV, wherein variants of said CTL epitope CVADYSVLYNSASFSTFKCY are CVADXSXLYNSASFSTXKCY, wherein each “X” is a variable amino acid and comprises any of the proteinogenic 20 amino acids the standard genetic code.

4. The method of claim 1, wherein the SARS-CoV-2 viral antigen comprises a CTL epitope, and wherein the variable amino acids can be any naturally occurring amino acids.

5. The method of claim 1, wherein the composition is administered to the subject prophylactically, optionally at a dose from 100 μg to 1 mg of isolated peptides, optionally at weekly intervals.

6. The method of claim 1, wherein the subject has a COVID-19 associated disease and wherein the composition is administered to the subject therapeutically, optionally at a dose from 100 μg to 1 mg of isolated peptides, optionally at weekly intervals.

7. The method of claim 1, wherein the total number of different peptides in the library ranges from 20 to 8,000, optionally, wherein the total number of different peptides in the library is 87.

8. The method of claim 1, wherein said each variable amino acid is
(i) selected from one or more of the group consisting of Alanine, Cysteine, Aspartate, Glutamate, Phenylalanine, Histidine, Isoleucine, Leucine, Asparagine, Glutamine, Arginine, Threonine, Valine and Tryptophan; or
(ii) selected from one or more of the group consisting of Aspartate, Phenylalanine, Isoleucine, Lysine, Leucine, Methionine, Asparagine, Glutamine, Serine, Threonine, Valine and Tyrosine; or
(iv) selected from one or more of the group consisting of Alanine, Aspartate, Glutamate, Phenylalanine, Glycine, Histidine, Isoleucine, Leucine, Asparagine, Proline, Glutamine, Arginine, Serine, Threonine, Valine and Tyrosine.

9. The method of claim 1, wherein prophylactically administering the variable epitope library vaccine composition, or nucleic acid encoding said peptides, results in
(i) an increased proliferation of splenocytes of said subject relative to that resulting from administering COVID-19-peptides or nucleic acid encoding said peptides and/or
(ii) an immune response of said subject comprising an increased number of CD8+IFN-γ+ cells which recognize variant COVID-19-derived CTL epitopes than in the immune response resulting from administering COVID-19-peptides or nucleic acid encoding said peptides.

10. A method of identifying a set of peptides for the treatment and/or prevention of disease in a subject resulting from infection with SARS-CoV-2, wherein the set of peptides comprises one or more peptides comprising (i) a T cell epitope of an antigen expressed in said subject and/or (ii) variants of said T-cell epitope, comprising:
(a) generating a combinatorial variable epitope library (VEL) wherein said VEL comprises a plurality of peptides, each said peptide comprising a T cell epitope or variant thereof, wherein the length of each said T cell epitope or variant thereof, ranges from 8 to 11 amino acids, wherein the amino acid residues at MHC class I-anchor positions of said T cell epitope and its variant are identical, and wherein the sequence of said T cell epitope and said variant thereof differ in at least two amino acid residues,
(b)
(i) incubating said T cell epitope or a variant thereof, with peripheral blood mononuclear cells (PBMCs) from a healthy individual (or a population of healthy individuals) under conditions suitable for inducing proliferation of PBMCs;
(ii) incubating said T cell epitope or variant thereof, with PBMCs from said individual infected with SARS-CoV-2 under conditions suitable for inducing proliferation of PBMCs, wherein said afflicted individual has a MHC Class I haplotype which is similar to the MHC Class I haplotype of said healthy individual,
(iii) comparing the proliferation of said T cell epitope and of each said variant thereof, in step (b)(i) versus step (b)(ii), thereby identifying three peptide groups:
(a) Group I—peptides which induce proliferation of PBMCs of said afflicted individual and in said healthy population;
(b) Group II—peptides which induce proliferation of PBMCs of said afflicted individual but not in said healthy population; and
(c) Group III—peptides which do not induce proliferation of PBMCs of said afflicted individual but induce proliferation in said healthy population
wherein each said peptide Group, or a combination of two or more of Groups I, II, and/or III, identifies a set of peptides for treatment against said disease or condition afflicting said individual.

11. The method of claim 10, wherein said method comprises chemical synthesis of said peptides, wherein optionally, the chemical synthesis is performed in the wells of a 96 well plate.

12. The method of claim 10, wherein when the amino acid sequence of said T cell epitope and its variant thereof differ at only two amino acid residues, the VEL comprises at least 100 variant peptides, or wherein when the amino acid sequence of said T cell epitope and its variant thereof differ at only three amino acid residues, the VEL comprises at least 1000 variant peptides.

13. The method of claim 12, wherein said variants are selected randomly.

14. The method of claim 10, wherein said T cell epitope comprises an amino acid sequence selected from the group consisting of: IVNSVLLFLAFVVFLLVTLAILTAL, AILTALRLCAYCCNIVNVSLVKPSFYVY, FLWLLWPVTLACFVLAAVYRI, TVATSRTLSYYKL, SASAFFGMSRIGMEVTPSGTWLTYTGAIKL, YTMADLVYAL, SMMGFKMNY, FLMSFTVLCLTPVY, KLNDLCFTNVYADSFVIRGDEVRQIAPGQTGKIADYNYKL, YIWLGFIAGLIAIV, CVADYSVLYNSASFSTFKCY and FERDISTEIYQAGSTPCNGVEGFNCYFPLQS.

15. The method of claim 14, wherein variants of said CTL epitope IVNSVLLFLAFVVFLLVTLAILTAL are IVNSVLXFLAFXVFLLVTLXILTAL, wherein variants of said CTL epitope AILTALRLCAYCCNIVNVSLVKPSFYVY are AILTXLRLCAYXCNIVXVSLVKPXFYVY; wherein variants of said CTL epitope FLWLLWPVTLACFVLAAVYRI are FLWXLXPVTLXCFVLXAVYRI, wherein variants of said CTL epitope TVATSRTLSYYKL are TVXTSRXLSXYKL, wherein variants of said CTL epitope SASAFFGMSRIGMEVTPSGTWLTYTGAIKL are SAXAFXGMSRXGMEVTPSGTWLTYXGXIKL, wherein variants of said CTL epitope YTMADLVYAL are YTXADXVXAL, wherein variants of said CTL epitope SMMGFKMNY are SMXGXKXNY, wherein variants of said CTL epitope FLMSFTVLCLTPVY are FLMXFXVLCXTPVY, wherein variants of said CTL epitope KLNDLCFTNVYADSFVIRGDEVRQIAPGQTGKIADYNYKL are KLNDLXFXNVYADSFVIRGDEXRQIAPGQTGKIADXNXKL, wherein variants of said CTL epitope YIWLGFIAGLIAIV are YIWLXFIXGXIAIV, wherein variants of said CTL epitope CVADYSVLYNSASFSTFKCY are CVADXSXLYNSASFSTXKCY, wherein each “X” is a variable amino acid and comprises any of the 20 proteinogenic amino acids the standard genetic code.

16. The method of claim 10, further comprising immunization of the afflicted individual with a formulation comprising at least one or with the mixture of up to 100 variant peptides identified in step (b) and pharmaceutically acceptable carrier.

17. The method of claim 10, wherein the sets of peptide epitopes of said combinatorial variable epitope library (VEL) are expressed by one or more of the group consisting of plasmid DNA, a viral vector and a microorganism.

18. The method of claim 17, wherein the sets of peptide epitopes of said combinatorial variable epitope library (VEL) are present at the surface of said microorganism, wherein said microorganism is selected from the group consisting of bacteriophage, yeast and bacteria.

19. The method of claim 10, wherein the sets of peptide epitopes of said combinatorial variable epitope library (VEL), are expressed on the surface of insect cells in combination with an MHC class I molecule.

20. The method of claim 10, wherein said plurality of peptides comprises three or more peptides.


SEQUENCE LISTING (24 sequences)

SEQ ID NO: 1 - protein, 25 aa, Artificial Sequence
  2019-nCoV env protein fragment
IVNSYLLFLAFVVFLLVTLAILTAL

SEQ ID NO: 2 - protein, 25 aa, Artificial Sequence
  CoVV1 VEL Vaccine
  MISC_FEATURE (7)..(7): X = any out of 20 proteinogenic amino acids
  MISC_FEATURE (12)..(12): X = any out of 20 proteinogenic amino acids
  MISC_FEATURE (20)..(20): X = any out of 20 proteinogenic amino acids
IVNSVLXFLAFXVFLLVTLXILTAL

SEQ ID NO: 3 - protein, 28 aa, Artificial Sequence
  2019-nCoV Env protein fragment
AILTALRLCAYCCNIVNVSLVKPSFYVY

SEQ ID NO: 4 - protein, 28 aa, Artificial Sequence
  CoVV2 VEL Vaccine
  misc_feature (5)..(5): Xaa can be any naturally occurring amino acid
  misc_feature (12)..(12): Xaa can be any naturally occurring amino acid
  misc_feature (17)..(17): Xaa can be any naturally occurring amino acid
  misc_feature (24)..(24): Xaa can be any naturally occurring amino acid
AILTXLRLCAYXCNIVXVSLVKPXFYVY

SEQ ID NO: 5 - protein, 21 aa, Artificial Sequence
  2019-nCoV Membrane glycoprotein fragment
FLWLLWPVTLACFVLAAVYRI

SEQ ID NO: 6 - protein, 23 aa, Artificial Sequence
  CoVV3 VEL Vaccine
  MISC_FEATURE (5)..(5): X = any out of 20 proteinogenic amino acids
  MISC_FEATURE (13)..(13): X = any out of 20 proteinogenic amino acids
  MISC_FEATURE (18)..(18): X = any out of 20 proteinogenic amino acids
FLINXLICPVTLXCFVLXAVYRI

SEQ ID NO: 7 - protein, 13 aa, Artificial Sequence
  2019-nCoV Membrane glycoprotein fragment
TVATSRTLSYYKL

SEQ ID NO: 8 - protein, 13 aa, Artificial Sequence
  CoVV4  VEL Vaccine
  MISC_FEATURE (3)..(3): X = any out of 20 proteinogenic amino acids
  MISC_FEATURE (7)..(7): X = any out of 20 proteinogenic amino acids
  MISC_FEATURE (10)..(10): X = any out of 20 proteinogenic amino acids
TVXTSRXLSXYKL

SEQ ID NO: 9 - protein, 30 aa, Artificial Sequence
  2019-nCoV Nucleocapsid protein fragment
SASAFFGMSRIGMEVTPSGTWLTYTGAIKL

SEQ ID NO: 10 - protein, 30 aa, Artificial Sequence
  CoVV5 VEL Vaccine
  MISC_FEATURE (3)..(3): X = any out of 20 proteinogenic amino acids
  MISC_FEATURE (6)..(6): X = any out of 20 proteinogenic amino acids
  MISC_FEATURE (11)..(11): X = any out of 20 proteinogenic amino acids
  misc_feature (25)..(25): Xaa can be any naturally occurring amino acid
  misc_feature (27)..(27): Xaa can be any naturally occurring amino acid
SAXAFXGMSRXGMEVTPSGTWLTYXGXIKL

SEQ ID NO: 11 - protein, 10 aa, Artificial Sequence
  2019-nCoV ORF1ab protein fragment
YTMADLVYAL

SEQ ID NO: 12 - protein, 10 aa, Artificial Sequence
  CoVV6 VEL Vaccine
  MISC_FEATURE (3)..(3): X = any out of 20 proteinogenic amino acids
  MISC_FEATURE (6)..(6): X = any out of 20 proteinogenic amino acids
  MISC_FEATURE (8)..(8): X = any out of 20 proteinogenic amino acids
YTXADXVXAL

SEQ ID NO: 13 - protein, 9 aa, Artificial Sequence
  2019-nCoV ORF1ab protein fragment
SMMGFKMNY

SEQ ID NO: 14 - protein, 9 aa, Artificial Sequence
  CoVV7 VEL Vaccine
  MISC_FEATURE (3)..(3): X = any out of 20 proteinogenic amino acids
  MISC_FEATURE (5)..(5): X = any out of 20 proteinogenic amino acids
  MISC_FEATURE (7)..(7): X = any out of 20 proteinogenic amino acids
SMXGXKXNY

SEQ ID NO: 15 - protein, 14 aa, Artificial Sequence
  2019-nCoV ORF1ab protein fragment
FLMSFTVLCLTPVY

SEQ ID NO: 16 - protein, 14 aa, Artificial Sequence
  CoVV8 VEL Vaccine
  MISC_FEATURE (4)..(4): X = any out of 20 proteinogenic amino acids
  MISC_FEATURE (6)..(6): X = any out of 20 proteinogenic amino acids
  MISC_FEATURE (10)..(10): X = any out of 20 proteinogenic amino acids
FLMXFXVLCXTPVY

SEQ ID NO: 17 - protein, 40 aa, Artificial Sequence
  2019-nCoV spike protein fragment
KLNDLCFTNVYADSFVIRGDEVRQIAPGQTGKIADYNYKL

SEQ ID NO: 18 - protein, 40 aa, Artificial Sequence
  CoVV9 VEL Vaccine
  MISC_FEATURE (6)..(6): X = any out of 20 proteinogenic amino acids
  MISC_FEATURE (8)..(8): X = any out of 20 proteinogenic amino acids
  MISC_FEATURE (22)..(22): X = any out of 20 proteinogenic amino acids
  MISC_FEATURE (36)..(36): X = any out of 20 proteinogenic amino acids
  misc_feature (38)..(38): Xaa can be any naturally occurring amino acid
KLNDLXFXNVYADSFVIRGDEXRQIAPGQTGKIADXNXKL

SEQ ID NO: 19 - protein, 14 aa, Artificial Sequence
  2019-nCoV spike protein fragment
YIWLGFIAGLIAIV

SEQ ID NO: 20 - protein, 14 aa, Artificial Sequence
  CoVV10 VEL Vaccine
  MISC_FEATURE (5)..(5): X = any out of 20 proteinogenic amino acids
  MISC_FEATURE (8)..(8): X = any out of 20 proteinogenic amino acids
  MISC_FEATURE (10)..(10): X = any out of 20 proteinogenic amino acids
YIWLXFIXGXIAIV

SEQ ID NO: 21 - protein, 20 aa, Artificial Sequence
  2019-nCoV spike protein fragment
CVADYSVLYNSASFSTFKCY

SEQ ID NO: 22 - protein, 20 aa, Artificial Sequence
  CoVV11 VEL Vaccine
  MISC_FEATURE (5)..(5): X = any out of 20 proteinogenic amino acids
  MISC_FEATURE (7)..(7): X = any out of 20 proteinogenic amino acids
  MISC_FEATURE (17)..(17): X = any out of 20 proteinogenic amino acids
CVADXSXLYNSASFSTXKCY

SEQ ID NO: 23 - protein, 31 aa, Artificial Sequence
  2019-nCoV spike protein fragment
FERDISTEIYQAGSTPCNGVEGFNCYFPLQS

SEQ ID NO: 24 - protein, 31 aa, Artificial Sequence
  COV12  VEL Vaccine
  MISC_FEATURE (9)..(9): X = any out of 20 proteinogenic amino acids
  MISC_FEATURE (12)..(12): X = any out of 20 proteinogenic amino acids
  MISC_FEATURE (14)..(15): X = any out of 20 proteinogenic amino acids
  MISC_FEATURE (20)..(20): X = any out of 20 proteinogenic amino acids
  MISC_FEATURE (22)..(22): X = any out of 20 proteinogenic amino acids
FERDISTEXYQXGXTPCNGXEXFNCYFPLQS